(19) United States
(12) Patent Application Publication
(10) Pub. No.: US 2025/0140400 A1
(43) Pub. Date: May 1, 2025

(54) REMOTE IMAGE DIAGNOSIS COMPREHENSIVE SUPPORT APPARATUS, METHOD OF COMPREHENSIVE SUPPORT FOR REMOTE DIAGNOSTIC IMAGING AND PROGRAM

(71) Applicant: KABUSHIKI KAISYA DOCTOR BANK, Osaka-shi, Osaka (JP)

(72) Inventor: Hiroyuki KAGEYAMA, Osaka-shi, Osaka (JP)

(21) Appl. No.: 18/714,009

(22) PCT Filed: Nov. 2, 2023
(86) PCT No.: PCT/JP2023/039585

§ 371 Date: May 28, 2024

(30) Foreign Application Priority Data
Oct. 27, 2023 (JP) 2023-184580

(51) Int. Cl. G16H 40/67; G16H 30/20; G16H 30/40; G16H 50/20

(52) U.S. Cl. CPC G16H 40/67; G16H 30/20; G16H 30/40; G16H 50/20

(57) ABSTRACT

A remote image diagnosis comprehensive support apparatus that requests external interpretation of medical images via a predetermined network contains a request acceptance section that accepts a request; a payment processing section that executes payment by the requester and executes transfer to an acceptant; a storage area creation section that creates a storage area for securely storing information on the predetermined network; an access right granting section that grants access rights to the storage area to the requester and the acceptant, and notifies the requester and the acceptant of addresses for accessing the storage area; and a storage information management section that manages inputting and outputting of information into and out of the storage area by the requester and the acceptant.

### TECHNICAL FIELD

The description relates to a remote image diagnosis comprehensive support apparatus, a method of comprehensive support for remote diagnostic imaging and a program.

### BACKGROUND ART

In the past, with the development of X-ray technology and the advancement and spread of technologies such as CT (Computed Tomography), MRI (Magnetic Resonance Imaging) and PET (Positron Emission Tomography), and so forth, a burden placed on doctors (hereinafter also referred to as clinicians) who clinically diagnose patients based on an images (hereinafter referred to as medical images) taken by these medical inspection apparatuses is increasing.

In view of such situation, in recent years, there has been an increase in the number of cases in which clinicians request specialized doctors (hereinafter also referred to as interpretation doctors) to interpret medical images, with the aim of reducing their own burden and making more accurate diagnoses.

Conventionally, when a clinician who directly diagnose a patient, a medical institution to which the clinician belongs, or the like, (hereinafter, these are also collectively referred to as requesters) requests an interpretation doctor, a professional image interpretation company, or the like, (hereinafter, these are also collectively referred to as acceptants) to interpret medical images, a patient personal information is provided to the acceptant along with the medical images. Therefore, in the past, in consideration of the confidentiality of the information, a dedicated line was used to connect the requester and the acceptant, and the information was exchanged via this dedicated line.

### CITATION LIST

### Patent Literature

### [PTL 1]

Japanese Laid-Open Patent Publication No. 2023-102155

### [PTL 2]

Japanese Laid-Open Patent Publication No. 2023-114242

### SUMMARY OF INVENTION

### Technical Problem

However, with the conventional system that connects the requester and the acceptant through a dedicated line, there are high hurdles in terms of costs such as initial costs, maintenance costs after introduction, and so forth, ease of operation after introduction, etc., especially for doctors, for instance, who are planning to start their own practice as an interpretation doctor, and therefore, even if they have the skills to interpret medical images, many doctors are reluctant to actively participate in such medical care as interpretation doctors.

In addition, in cases where a professional image interpretation company acts as a point of contact to accept requests, at the professional image interpretation company, a lot of efforts and intermediate costs occur, such as distributing the image interpretation system to each interpretation doctor, allocating work to each interpretation doctor, confirming billing and payment to requesters, calculating and paying salaries to each interpretation doctor, or the like. As a result, there are high hurdles for new entrants, and it has been difficult to create an environment in which more doctors can participate as interpretation doctors.

Therefore, the present disclosure has been made in view of the above-mentioned problems, and the purpose of the present disclosure is to provide a remote image diagnosis comprehensive support apparatus, a method of comprehensive support for remote diagnostic imaging and a program that enable more doctors to participate in medical care as interpretation doctors while maintaining information confidentiality.

### Solution to Problem

A remote image diagnosis comprehensive support apparatus according to an embodiment of the present disclosure that requests interpretation of medical images in DICOM (digital imaging and communication in medicine) format to an external party via a predetermined network, comprises a request acceptance section that accepts a request for an image interpretation from a requester; a payment processing section that executes payment by the requester regarding the request and executes transfer to an acceptant who has uploaded an interpretation report which is a deliverable regarding the request; a storage area creation section that creates a storage area for securely storing highly confidential request information including a medical image on the predetermined network; an access right granting section that grants an access right to the storage area to the requester and notifies the requester of an address for accessing the storage area, and that grants an access right to the storage area to the acceptant who accepted the request and notifies the acceptant of the address for accessing the storage area; and a storage information management section that stores the request information uploaded by the requester in the storage area and downloads the request information in the storage area to the acceptant on demand from the acceptant, and that stores the interpretation report regarding the medical image uploaded by the acceptant in the storage area and downloads the interpretation report in the storage area to the requester on demand from the requester.

A method of comprehensive support for remote diagnostic imaging according to an embodiment of the present disclosure that requests interpretation of medical images in DICOM (digital imaging and communication in medicine) format to an external party via a predetermined network, comprises accepting a request for an image interpretation from a requester; executing payment by the requester regarding the request; creating a storage area for securely storing highly confidential request information including a medical image on the predetermined network; granting an access right to the storage area to the requester; notifying the requester of an address for accessing the storage area; storing the request information uploaded by the requester in the storage area; granting an access right to the storage area to the acceptant who accepted the request; notifying the acceptant of the address for accessing the storage area; downloading the request information in the storage area to the acceptant on demand from the acceptant; storing an interpretation report regarding the medical image uploaded by the acceptant in the storage area; executing transfer to the acceptant uploaded the interpretation report; and downloading the interpretation report in the storage area to the requester on demand from the requester.

A program according to an embodiment of the present disclosure for operating processor that requests interpretation of medical images in DICOM (digital imaging and communication in medicine) format to an external party via a predetermined network, the program includes instructions which, when executed by the processor, cause the processer to perform operations comprising: accepting a request for an image interpretation from a requester; executing payment by the requester regarding the request; creating a storage area for securely storing highly confidential request information including a medical image on the predetermined network; granting an access right to the storage area to the requester; notifying the requester of an address for accessing the storage area; storing the request information uploaded by the requester in the storage area; granting an access right to the storage area to the acceptant who accepted the request; notifying the acceptant of the address for accessing the storage area; downloading the request information in the storage area to the acceptant on demand from the acceptant; storing an interpretation report regarding the medical image uploaded by the acceptant in the storage area; executing transfer to the acceptant uploaded the interpretation report; and downloading the interpretation report in the storage area to the requester on demand from the requester.

### BRIEF DESCRIPTION OF DRAWINGS

FIG. 1 is a schematic diagram illustrating an example of a schematic configuration of a remote image diagnosis comprehensive support system for realizing a remote image diagnosis comprehensive support service according to a first embodiment of the present disclosure.

FIG. 2 is a functional block diagram illustrating a schematic configuration example of an acceptance management server according to the first embodiment of the present disclosure.

FIG. 3 is a diagram illustrating an example of a request management table according to the first embodiment of the present disclosure.

FIG. 4 is a functional block diagram illustrating a schematic configuration example of an information storage server according to the first embodiment of the present disclosure.

FIG. 5 is a functional block diagram illustrating a schematic configuration example of a payment server according to the first embodiment of the present disclosure.

FIG. 6 is a functional block diagram illustrating a schematic configuration example of a user management server according to the first embodiment of the present disclosure.

FIG. 7 is a diagram illustrating an example of items managed in the requester DB according to the first embodiment of the present disclosure.

FIG. 8 is a diagram illustrating an example of items managed in the acceptant DB according to the first embodiment of the present disclosure.

FIG. 9 is a sequence diagram illustrating an example of an operation of the remote image diagnosis comprehensive support system according to the first embodiment of the present disclosure (Part 1).

FIG. 10 is a sequence diagram illustrating the example of the operation of the remote image diagnosis comprehensive support system according to the first embodiment of the present disclosure (Part 2).

FIG. 11 is a sequence diagram illustrating the example of the operation of the remote image diagnosis comprehensive support system according to the first embodiment of the present disclosure (Part 3).

FIG. 12 is a sequence diagram illustrating the example of the operation of the remote image diagnosis comprehensive support system according to the first embodiment of the present disclosure (Part 4).

FIG. 13 is a sequence diagram illustrating the example of the operation of the remote image diagnosis comprehensive support system according to the first embodiment of the present disclosure (Part 5).

FIG. 14 is a sequence diagram illustrating the example of the operation of the remote image diagnosis comprehensive support system according to the first embodiment of the present disclosure (Part 6).

FIG. 15 is a diagram illustrating an example of a request plan selection screen according to the first embodiment of the present disclosure.

FIG. 16 is a diagram illustrating an example of a login screen according to the first embodiment of the present disclosure.

FIG. 17 is a diagram illustrating an example of a new requester registration screen according to the first embodiment of the present disclosure.

FIG. 18 is a diagram illustrating an example of an image interpretation request registration screen for diagnostic purposes according to the first embodiment of the present disclosure.

FIG. 19 is a diagram illustrating an example of an image interpretation request registration screen for research purposes according to the first embodiment of the present disclosure.

FIG. 20 is a diagram illustrating an example of an image interpretation request registration screen for verification purposes according to the first embodiment of the present disclosure.

FIG. 21 is a diagram illustrating an example of request information according to the first embodiment of the present disclosure (Part 1).

FIG. 22 is a diagram illustrating the example of the request information according to the first embodiment of the present disclosure (Part 2).

FIG. 23 is a diagram illustrating an example of a registration information management screen according to the first embodiment of the present disclosure.

FIG. 24 is a diagram illustrating an example of a request summary confirmation screen according to the first embodiment of the present disclosure.

FIG. 25 is a diagram illustrating an example of an interpretation report according to the first embodiment of the present disclosure (part 1).

FIG. 26 is a diagram illustrating the example of the interpretation report according to the first embodiment of the present disclosure (part 2).

FIG. 27 is a diagram illustrating the example of the interpretation report according to the first embodiment of the present disclosure (part 3).

FIG. 28 is a diagram illustrating another example of the interpretation report according to the first embodiment of the present disclosure (Part 1).

FIG. 29 is a diagram illustrating the another example of the interpretation report according to the first embodiment of the present disclosure (Part 2).

FIG. 30 is a diagram illustrating the another example of the interpretation report according to the first embodiment of the present disclosure (Part 3).

FIG. 31 is a diagram illustrating the another example of the interpretation report according to the first embodiment of the present disclosure (Part 4).

FIG. 32 is a diagram illustrating the another example of the interpretation report according to the first embodiment of the present disclosure (Part 5).

FIG. 33 is a diagram illustrating the another example of the interpretation report according to the first embodiment of the present disclosure (Part 6).

FIG. 34 is a schematic diagram illustrating a schematic configuration example of a remote image diagnosis comprehensive support system for realizing a remote image diagnosis comprehensive support service according to a second embodiment of the present disclosure.

FIG. 35 is a sequence diagram illustrating an example of an operation of the remote image diagnosis comprehensive support system according to the second embodiment of the present disclosure.

FIG. 36 is a sequence diagram illustrating an example of an operation of the remote image diagnosis comprehensive support system according to a modification of the second embodiment of the present disclosure.

FIG. 37 is a diagram illustrating a schematic configuration example of a request support system for realizing a request support service according to a third embodiment of the present disclosure.

FIG. 38 is a block diagram illustrating a schematic configuration example of each information processing device according to each embodiment of the present disclosure.

### DESCRIPTION OF EMBODIMENTS

In the following, a remote image diagnosis comprehensive support apparatus, a method of comprehensive support for remote diagnostic imaging and a program according to the present disclosure will be described in detail by illustrating several embodiments.

### First Embodiment

First, a first embodiment of the present disclosure will be described in detail with reference to drawings. In the present embodiment, a remote image diagnosis comprehensive support apparatus that supports clinicians (here, clinicians may include other doctors such as pathologists, internists, surgeons, or the like), medical institutions, or the like, (hereinafter also referred to as requesters) in requesting interpretation doctors and companies that provide remote image diagnosis support services to interpret medical images will be explained using an example.

Note that a predetermined contract may or may not exist between the requester and the acceptant for requesting and receiving requests for image interpretations. For example, a requester such as a clinician or a medical institution may request an unspecified interpretation doctor or an unspecified professional image interpretation company with whom no specific contract has been signed, or a specific interpretation doctor or a specific professional image interpretation company with whom a specific contract has been signed, for an image interpretation for each case, or for a set number of image interpretations on a regular basis (for example, once a week). That is, in the present embodiment can be applied to a system that mediates between a requester and an acceptant, regardless of the contents of a contract concluded between the requester and the acceptant.

The purposes of interpretations of medical images include diagnostic purposes for patients, research purposes at research institutions, and verification purposes for verifying evidence in the event of a medical problem or in a judicial autopsy, etc., and in the present embodiment, to simplify the explanation, a case of the diagnostic purposes for patients will be described as an example.

In the present embodiment, a case in which one or more requesters request interpretation of medical images for one acceptant is exemplified. In the following, for clarity, a case where an acceptant is an individual interpretation doctor will be exemplified. Moreover, a requester may be an individual doctor who directly diagnoses patients or his or her support staff, or may be a medical institution such as a hospital or research institute to which the doctor or the like belongs. In the following, for clarity, a case where an acceptant is a medical institution will be exemplified.

Here, “medical images” can be various image data or video data obtained by medical inspection apparatuses, such as X-ray images, CT images, PET-CT images, MRI images, echo images, endoscopic images, electrocardiograms, and pathological images. However, it is not limited thereto, and the medical image according to the present embodiment may include various image data and video data used by a doctor for diagnosis. As a data format of a medical image, a DICOM (Digital Imaging and Communication in Medicine) format will be exemplified in the following embodiments. However, the data format is not limited to this, and may be in various data formats of still images, video, etc., such as JPEG, TIFF, GIF, BMP, MP4, or the like.

### (System Configuration)

First, an example of a schematic configuration of a remote image diagnosis comprehensive support system according to the present embodiment will be described in detail with reference to drawings.

### /Remote Interpretation Total Support System 1

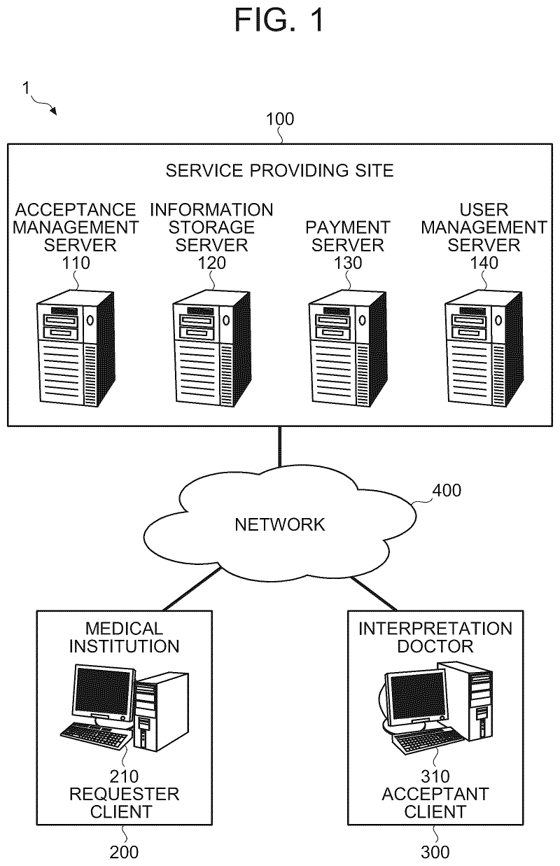

FIG. 1 is a schematic diagram illustrating an example of a schematic configuration of a remote image diagnosis comprehensive support system for realizing a remote image diagnosis comprehensive support service according to the present embodiment. As shown in FIG. 1, the remote image diagnosis comprehensive support system 1 includes various servers 110, 120, 130, and 140 that constitute a service providing site 100, a requester client 210 installed in a medical institution 200, and an acceptant client 310 owned by an interpretation doctor 300, which are connected to each other via a network 400 so as to be able to communicate with each other. Here, one or more of the various servers 110, 120, 130, and 140 that constitute the service providing site 100 may constitute a remote image diagnosis comprehensive support apparatus according to the present embodiment. The network 400 may be a wide area network such as the Internet or a mobile communication system, or may be a limited network such as a LAN (Local Area Network) or a WAN (Wide Area Network), or may be a combined network of these.

### //Medical Institution 200 and Requester Client 210

The requester client 210 may be, for example, a various personal computer (hereinafter also simply referred to as PC) such as a desktop type, a notebook type, or a tablet type installed in the medical institution 200 such as a hospital or a research institute. The requester client 210 has, for example, a communication function for connecting to the network 400 via a LAN built within the medical institution 200, and a web browser function for viewing web pages on the network 400 published by the service providing site 100. By accessing the service providing site 100 from the requester client 210, a doctor or his/her support staff belonging to the medical institution 200 can request an external party (for example, an interpretation doctor 300 in a remote location) to interpret medical images, and also, obtains the interpretation report created by the requested interpretation doctor 300. Note that the medical images may be medical images acquired by medical inspection apparatuses installed within the medical institution 200, or medical images acquired by medical inspection apparatuses installed at an external medical institution, research institute, or the like.

### //Interpretation Doctor 300 and Acceptant Client 310

The acceptant client 310 may be, for example, a various PC such as a desktop type, a notebook type, or a tablet type installed in an employment location or home of the interpretation doctor 300 (hereinafter referred to as the workplace). The acceptant client 310 has, for example, a communication function for connecting to the network 400 via a LAN built within the workplace, and a web browser function for viewing web pages on the network 400 published by the service providing site 100. By accessing the service providing site 100 from the acceptant client 310, an interpretation doctor 300 can accept a request registered in the service providing site 100, and obtains medical images to be interpreted. Then, the interpretation doctor 300 interprets the obtained medical images and stores an interpretation report created as a result on the service providing site 100.

### //Service Provider Site 100

The service providing site 100 may include, for example, an acceptance management server 110, an information storage server 120, a payment server 130, and a user management server 140. Note that, as shown in FIG. 1, the service providing site 100 may be composed of a plurality of servers (including cloud servers), or may be composed of a single server or two or more servers in which at least two of the servers 110, 120, 130 and 140 are integrated. Moreover, the service providing site 100 may be a dedicated EC site (also referred to as an in-house EC site) built for the purpose of providing the remote image diagnosis comprehensive support service according to the present embodiment, or may be a mall-type EC site that can be used by unspecified users for various purposes. For such the service providing site 100, an existing EC (Electronic Commerce) site such as Shopify (registered trademark) may be used. In such case, by using an EC site that provides not only requesting/accepting services but also payment services, it is possible to simplify the effort required of the requester and the acceptant.

### Acceptance Management Server 110

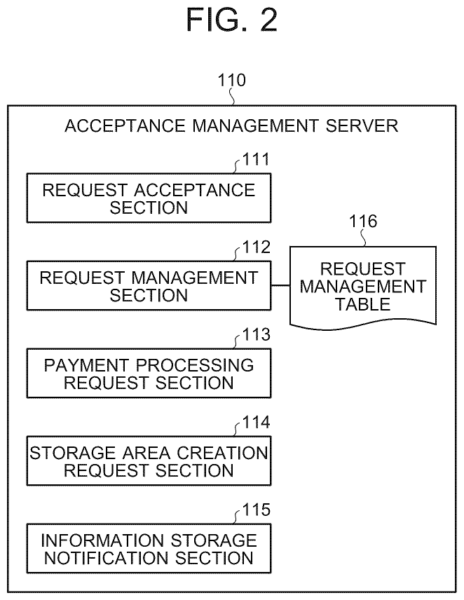

FIG. 2 is a functional block diagram illustrating a schematic configuration example of the acceptance management server according to the present embodiment. As shown in FIG. 2, the acceptance management server 110 includes a request acceptance section 111, a request management section 112, a payment processing request section 113, a storage area creation request section 114, and an information storage notification section 115. The acceptance management server 110 manages requests of image interpretations from the medical institution 200 that is the requester and requests accepted by the interpretation doctor 300 using a request management table 116 to be described later.

The request acceptance section 111 accepts requests from the requester (in this example, the medical institution 200).

The request management section 112 manages the requests accepted by the request acceptance section 111 by registering them in the request management table 116.

For each request, the payment processing request section 113 requests the payment server 130, which will be described later, to execute a payment processing (payment) by the requester and transfer processing to the acceptant.

When receiving a request from a requester, the storage area creation request section 114 requests the information storage server 120 to create a dedicated storage area.

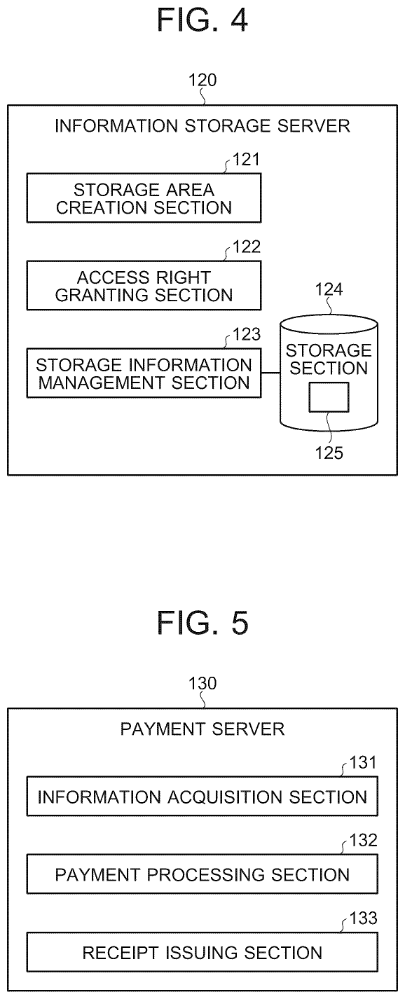

When information is stored in the storage area (see the storage area 125 in FIG. 4) secured in the information storage server 120, the information storage notification section 115 notifies the requester and/or the acceptant.

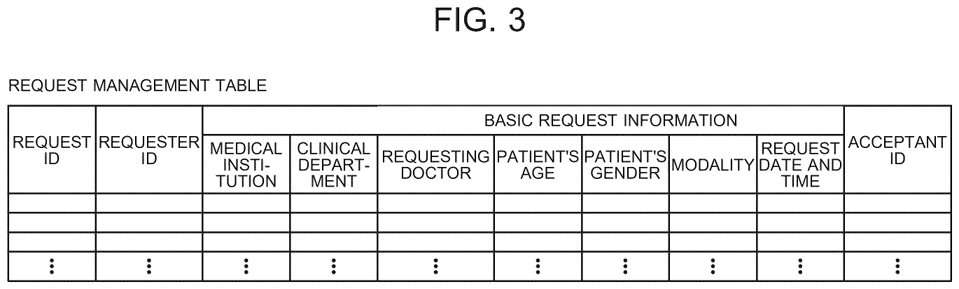

FIG. 3 is a diagram illustrating an example of a request management table according to the present embodiment. As shown in FIG. 3, in the request management table 116, for example, a request ID, a requester ID, basic request information, and an acceptant ID are managed in association with each other.

The request ID may be identification information for uniquely identifying a request, and may be a value assigned by the acceptance management server 110 each time a request is registered by a requester (in this example, the medical institution 200).

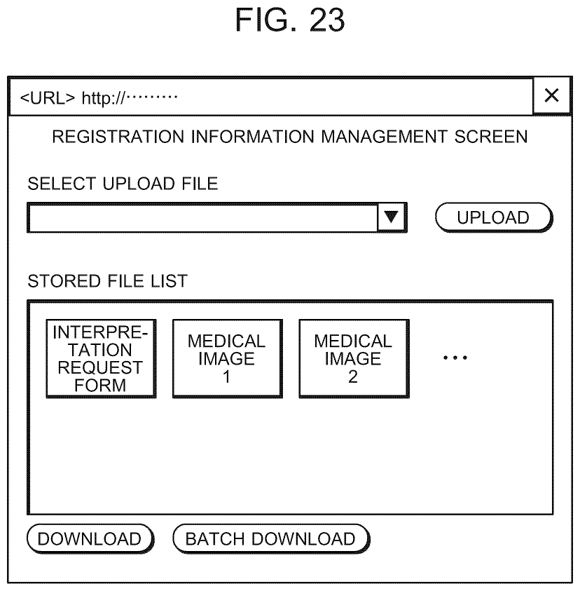

The requester ID may be identification information for uniquely identifying the requester. This requester ID may be a value given to the requester by the user management server 140, which will be described later, when the requester (in this example, the medical institution 200) is newly registered (including pre-registration) in the requester database (DB). The requester ID may be a value used as an access ID (also referred to as a login ID) when the requester accesses the storage area (see the storage area 125 in FIG. 4 and a registration information management screen in FIG. 23) to be described later.

The basic request information, which will be explained later, may include information such as a name of the medical institution 200 (if the requester is an individual doctor, a doctor name) that is the requester, a clinical department that diagnoses the medical images (in other words, a clinical department where a patient visits) that is the interpretation target, a requesting doctor, a patient's age, a patient's gender, modalities, a request date and time, and so forth. In addition to this, the request basic information may include a patient's name, a patient ID, a patient's date of birth, a clinical course, and points particularly desired for image interpretation. Here, since at least the patient's name, the patient ID, the patient's date of birth, the clinical course, and the points particularly desired for image interpretation are information that may correspond to the patient personal information, they may be registered in the request management table 116 after being encrypted. In such case, the encryption target may include at least one of the name of the medical institution 200 (or the name of the individual doctor), the clinical department that diagnoses the medical images (in other words, the clinical department where the patient visits), the requesting doctor, the patient's age, the patient's gender, the modalities, the request date and time, the request ID, the requester ID, and the acceptant ID.

The acceptant ID may be identification information for uniquely identifying the acceptant. This acceptant ID may be a value given to the acceptant by the user management server 140, which will be described later, when the acceptant (in this example, the interpretation doctor 300) is newly registered (including pre-registration) in the acceptant database (DB). The acceptant ID may be blank (Null) if an acceptant is undecided. The acceptant ID may be a value used as an access ID (also referred to as a login ID) when the acceptant accesses the storage area (see the registration information management screen in FIG. 23) to be described later.

### ///Information Storage Server 120

FIG. 4 is a functional block diagram illustrating a schematic configuration example of an information storage server according to the present embodiment. As shown in FIG. 4, the information storage server 120 includes a storage area creation section 121, an access right granting section 122, a storage information management section 123, and a storage section 124. The information storage server 120 stores various data such as medical images to be interpreted that are registered by the requester and interpretation reports that are registered by the acceptant as the interpretation results of the medical images.

The storage area creation section 121 creates, when receiving a creation of a storage area from the acceptance management server 110, a dedicated storage area (also referred to as data box or BOX) 125 in the storage section 124 that is associated with the request ID for each request.

The access right granting section 122 grants access rights to the storage area 125 associated with the request ID to the requester who made the request (in this example, the medical institution 200) and the acceptant who accepted the request (in this example, the interpretation doctor 300).

The storage information management section 123 manages and executes storing of various data in the storage area 125 such as medical images uploaded from requesters (in this example, the medical institution 200) and various data such as interpretation reports uploaded from acceptants (in this example, the interpretation doctor 300). Furthermore, the storage information management section 123 manages and executes reading out and downloading of various data from the storage area 125 such as medical images requested by acceptants and various data such as interpretation reports requested by the requesters.

Such information storage server 120 may be a server that can store information securely, such as a cloud storage, or the like, for instance, and as the information storage server 120, an existing cloud service or the like may be used.

### ///Payment Server 130

FIG. 5 is a functional block diagram illustrating a schematic configuration example of a payment server according to the present embodiment. As shown in FIG. 5, the payment server 130 includes an information acquisition section 131, a payment processing section 132, and a receipt issuing section 133, and manages and executes a payment processing such as payment by a requester and transfer to an acceptant. Furthermore, the receipt issuing section 133 issues a receipt to the requester or the medical institution (in this example, the medical institution 200) to which the requester belongs when the requester completes the payment. An existing payment site may be used as the payment server 130.

### ///User Management Server 140

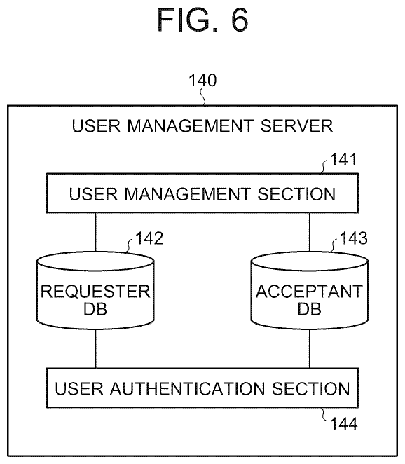

FIG. 6 is a functional block diagram illustrating a schematic configuration example of a user management server according to the present embodiment. As shown in FIG. 6, the user management server 140 includes a user management section 141, a requester DB 142, an acceptant DB 143, and a user authentication section 144, and executes management of information about the requester and the acceptant and login authentication of the requester and/or the acceptant to this system.

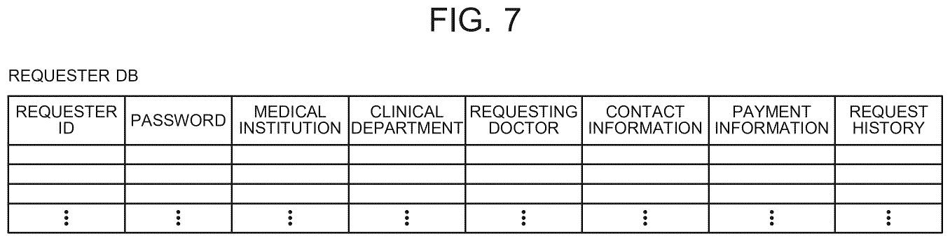

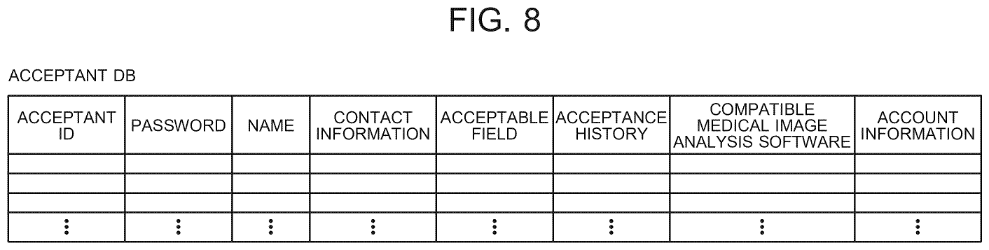

For instance, the user management server 140 manages the information about the requester using the requester DB 142 as illustrated in FIG. 7, and manages the information about the acceptant using the acceptant DB 143 as illustrated in FIG. 8. The user management section 141 performs processing such as registration, update and deletion of the information about the requester in the requester DB 142, and registration, update, and deletion of the information about the acceptant in the acceptant DB 143, notification of necessary information in response to requests from the acceptance management server 110, payment server 130, and the like.

The user authentication section 144 authenticates a user (a requester and/or an acceptant) who attempts to access this system using information registered in the requester DB 142 or the acceptant DB 143 in response to a request from the acceptance management server 110, for instance.

Note that information about a requester who can be a client for image interpretation may be registered in the user management server 140 in advance. By registering information about the requester in advance, in other words, by employing a registration system for individuals or medical institutions that can be requesters, it becomes possible not only to guarantee identities of the clients, but also to obtain benefits such as reducing the hassle of payment (payoff).

Similarly, the information about the acceptant may be registered in the user management server 140 in advance. By registering the information about the acceptant in advance, in other words, by employing a registration system for persons that can be acceptants, it becomes possible not only to guarantee identities of the acceptants, but also to obtain benefits such as reducing the hassle of payment (transfer).

### ////Contractee DataBase (DB)

FIG. 7 is a diagram illustrating an example of items managed in the requester DB according to the present embodiment. As shown in FIG. 7, in the requester DB 142, regarding each registered requester, information such as a requester ID, a password, a medical institution, a medical department, a name of a requesting doctor, contact information, payment information, and request history are managed in association with each other.

As described above, the requester ID may be identification information for uniquely identifying the requester. The requester ID may be used as a login ID for logging into this service.

The password may be a password set by the requester, and may be information used for authentication when logging into this service.

The medical institution may be the name of the medical institution to which the requesting doctor belongs (or identification information for uniquely identifying the medical institution).

The clinical department may be information about the medical department to which the requesting doctor belongs or the medical department that requests image interpretation. For example, if the requesting clinician is a doctor belonging to a radiology department, the radiology department may be specified, and if the requesting clinician is an internal medicine department, the internal medicine department may be specified.

Furthermore, if the requester has multiple clinical departments such as a general hospital, one or more medical departments that may request image interpretation may be specified.

The name of the requesting doctor may be information for identifying a natural person responsible for the request.

The contact information may be information necessary to contact the requester, such as address, telephone number, and e-mail address of the requester.

The payment information may be information used by the requester to make a payment (payoff), such as debit account information, credit card information, information about an online payment service such as PayPay (registered trademark), or the like. By managing the payment information of the requester in advance at the service providing site 100, it is possible to eliminate the trouble and risk of inputting payment information for each request. Note that the number of pieces of the payment information is not limited to one, and a plurality of pieces of the payment information may be registered.

The request history may be information about the number of requests for each field (for example, clinical department) in which the requester has requested image interpretation in the past.

### Contractor DataBase (DB)

FIG. 8 is a diagram illustrating an example of items managed in the acceptant DB according to the present embodiment. As shown in FIG. 8, in the acceptant DB 143, regarding each registered acceptant, information such as acceptant ID, password, name, contact information, acceptable field, acceptance history, compatible medical image analysis software, and account information are managed in association with each other.

As described above, the acceptant ID may be identification information for uniquely identifying the acceptant. The acceptant ID may be used as a login ID for logging into this service.

The password may be a password set by the acceptant, and may be information used for authentication when logging into this service.

The name may be a personal name if the acceptant is an individual, or an organization name if the acceptant is an organization such as a company or enterprise.

The contact information may be information necessary to contact the acceptant, such as address, telephone number, and e-mail address of the acceptant.

The acceptable field may be information about a field in which the acceptant can accept requests for image interpretation such as a medical department, for instance. For example, if the interpretation doctor 300 who is the acceptant is a doctor specializing in radiology, the radiology department may be specified, and if the acceptant is an internal medicine doctor, the internal medicine department may be specified. Furthermore, if the acceptant is available for multiple medical departments, multiple medical departments may be specified.

The acceptant record may be information about the number of requests accepted for each field (for example, clinical department) in which the acceptant has accepted requests for image interpretation in the past, an accuracy of image interpretation, or the like. Here, the accuracy of image interpretation may reflect evaluations by requesters.

The compatible medical image analysis software may be a list of medical image analysis software that can be used for image interpretation by the interpretation doctor 300 who is the acceptant. For example, when the interpretation doctor 300 can use one or more of medical image analysis software such as Osirix (registered trademark), DICOM Library, IMAIOS DICOM Viewer, PostDICOM, Voxelx, FViewer, etc., one or more of available multiple software may be listed.

The account information may be information for the acceptant to receive payment (transfer), such as information about a bank account for transfer or an online payment service. By managing the account information of the acceptant on the service providing site 100 in advance, it is possible to eliminate the trouble and risk of inputting account information for each acceptance. Note that the number of account information is not limited to one, and a plurality of account information may be registered.

### (Operation Example)

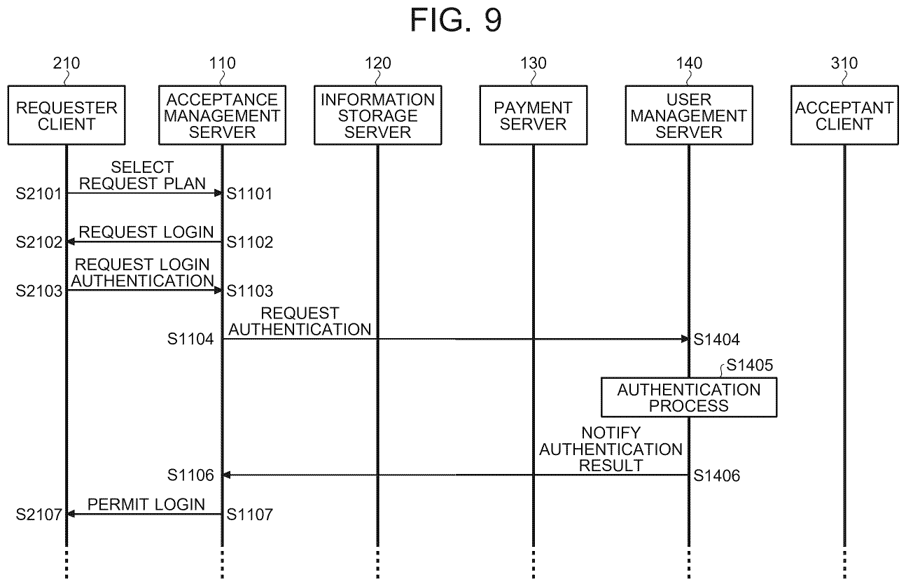

Next, an example of an operation of the remote image diagnosis comprehensive support system 1 according to the present embodiment will be described in detail with reference to the drawings. FIGS. 9 to 14 are sequence diagrams illustrating an example of the operation of the remote image diagnosis comprehensive support system according to the present embodiment.

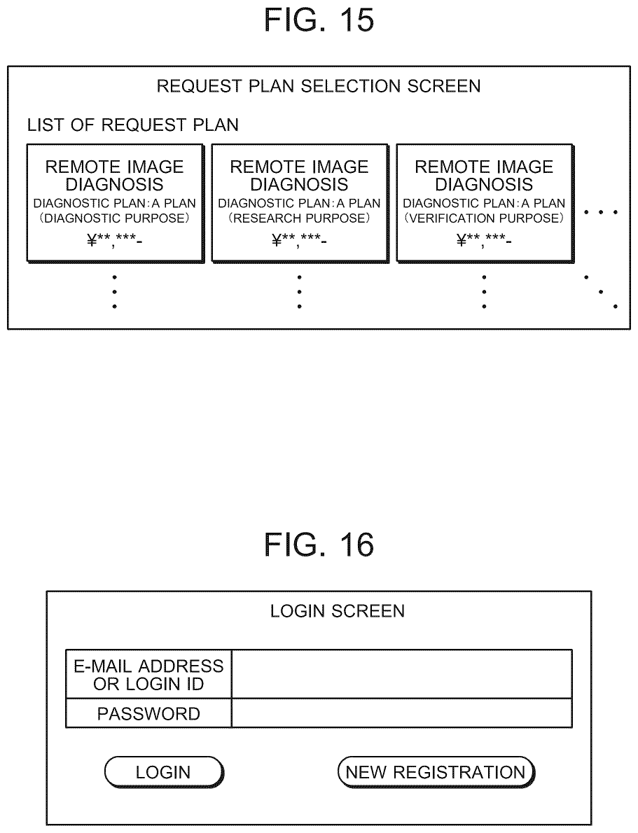

As shown in FIG. 9, in this operation, firstly, the medical institution 200 uses the requester client 210 to access a website (see a request plan selection screen shown in FIG. 15) provided by the acceptance management server 110 of the service providing site 100, and selects a request plan prepared in advance at the service providing site 100. (Step S2101 to S1101). The request plans may be products prepared in advance by the service providing site 100 based on general types of request contents (above-mentioned diagnostic purposes, research purposes, verification purposes, etc., for instance), fees, and the like.

Note that, the type of the request content may be categorized by purpose such as the above-mentioned diagnostic purpose, research purpose, or verification purpose, etc., or by clinical department, or by modality used to acquire the medical image, or by data format of the medical image to be interpreted (DICOM, JPEG, TIFF, GIF, BMP, MP4, etc.), or by application used for image interpretation (Osirix, DICOM Library, IMAIOS DICOM Viewer, PostDICOM, Voxelx, FViewer, etc.), or based on other factors, or by categories that are a combination of two or more of these, for instance.

In addition, the fee for the request plan is not limited to one request, but may be a fee for multiple requests such as five times, or a fee for each period such as one month or one year. When the fee is charged for each period, an upper limit on the number of requests per period (for example, 10 requests per month) may be set.

Here, the requested plan selection screen will be explained with reference to FIG. 15. FIG. 15 is a diagram illustrating an example of the request plan selection screen according to the present embodiment. As shown in FIG. 15, a list of one or more request plans may be displayed on the request plan selection screen. Each request plan is displayed as a selectable icon, and the request plan may be selected by the requester performing an operation such as double-clicking on the icon of the desired request plan.

After the request plan is selected, the request acceptance section 111 of the acceptance management server 110 displays a login screen for logging into this service (see FIG. 16) on a web browser of the requester client 210, and requests the medical institution 200 to log into this service (Step S1102 to S2102). In response, the medical institution 200 enters the login ID and the password assigned to itself on the login screen using the requester client 210, and requests login authentication from the acceptance management server 110 by selecting the “LOGIN” button (steps S2103 to S1103).

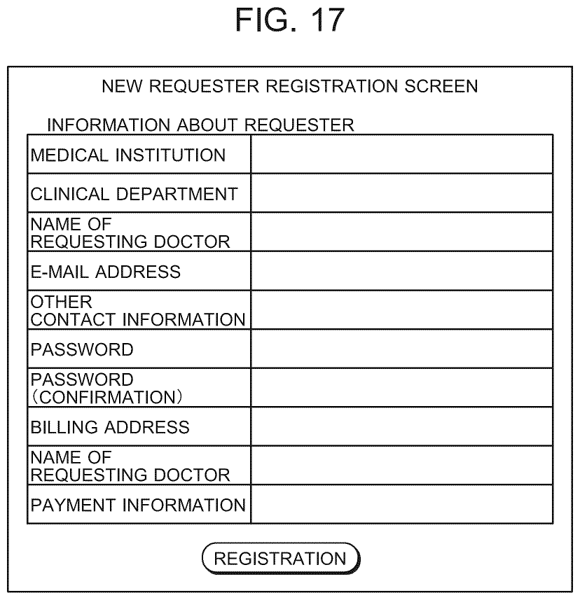

Note that, as described above, the requester ID given when the medical institution 200 registers as the requester may be used as the login ID. Alternatively, an e-mail address may be used instead of the login ID. Furthermore, if the medical institution 200 is not registered in this service as a requester, the request acceptance section 111 of the acceptance management server 110 may display a new requester registration screen as shown in FIG. 17 on the requester client 210, and register the medical institution 200 as a requester in the requester DB 142 by having the medical institution 200 input necessary information.

Next, the request acceptance section 111 of the acceptance management server 110 notifies the user management server 140 of the login information (login ID and password) entered by the medical institution 200 and requests the medical institution 200 to perform authentication processing (steps S1104 to S1404). In response, the user authentication section 144 of the user management server 140 authenticates whether or not the requester is a registered authorized requester by comparing the notified login information with login information (login ID and password) registered in the requester DB 142 (step S1405).

Next, the user authentication section 144 notifies the acceptance management server 110 of the authentication result (steps S1406 to S1106). In response, when the user authentication is successful, the request acceptance section 111 of the acceptance management server 110 permits the medical institution 200 (specifically, the requester client 210) to log in (steps S1107 to S2107). On the other hand, when the login authentication fails in step S1405, the login request (steps S1102 to 2102) may be executed again.

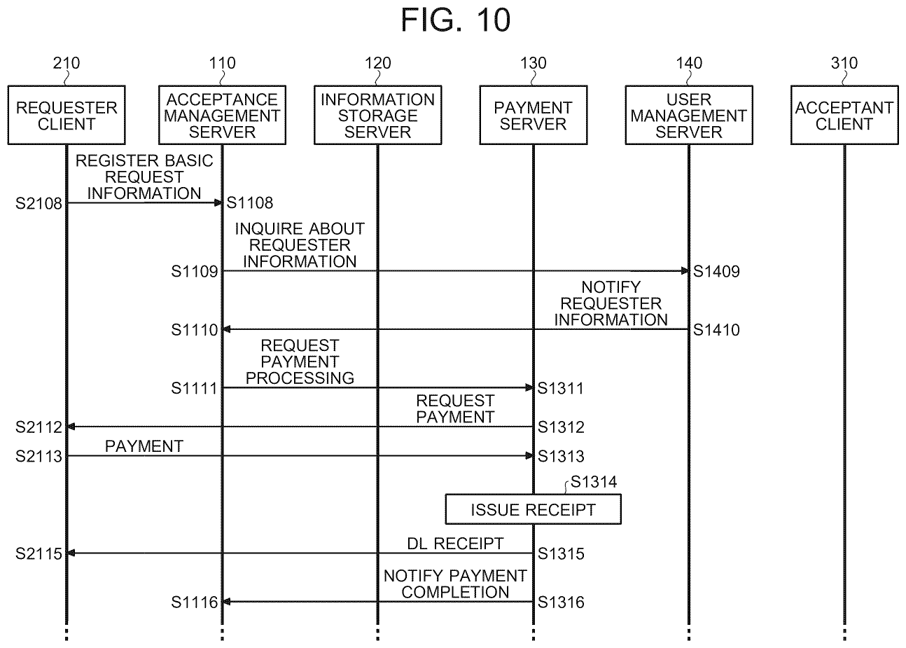

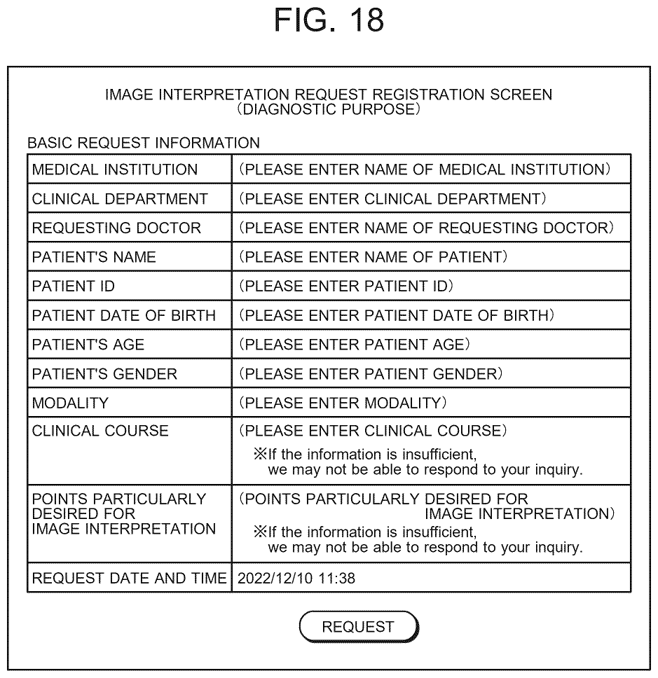

When the login is permitted for the requester client 210, as shown in FIG. 10, the requester client 210 displays a screen (see an image interpretation request registration screen in FIG. 18) for allowing the medical institution 200 to enter basic information about contents of the request (basic request information), and the basic request information is registered by the medical institution 200 according to this image interpretation request registration screen (steps S2108 to S1108). The image interpretation request registration screen may be published on the network 400 generated by the request acceptance section 111 of the acceptance management server 110, for example.

Here, the request basic information that the medical institution 200 registers when making a request will be explained using the image interpretation request registration screen for entering this request basic information. FIG. 18 is a diagram illustrating an example of the image interpretation request registration screen for diagnostic purposes according to the present embodiment. The image interpretation request registration screen has fields for entering a name of a requesting medical institution (if the requester is an individual doctor, a doctor name), a clinical department, a name of a requesting doctor, a patient's name, a patient ID, a patient's date of birth, a patient's age, a patient's gender, a modality, a clinical course, and points particularly desired for image interpretation. Note that the modality may be information about a type of medical testing device that acquired the medical image to be interpreted. This modality may include information about a file format of the medical image. The request date and time may be automatically set when a predetermined action is performed by the requester, such as when the requester (in this example, the medical institution 200) accesses the image interpretation request registration screen or when the requester clicks a “REQUEST” button.

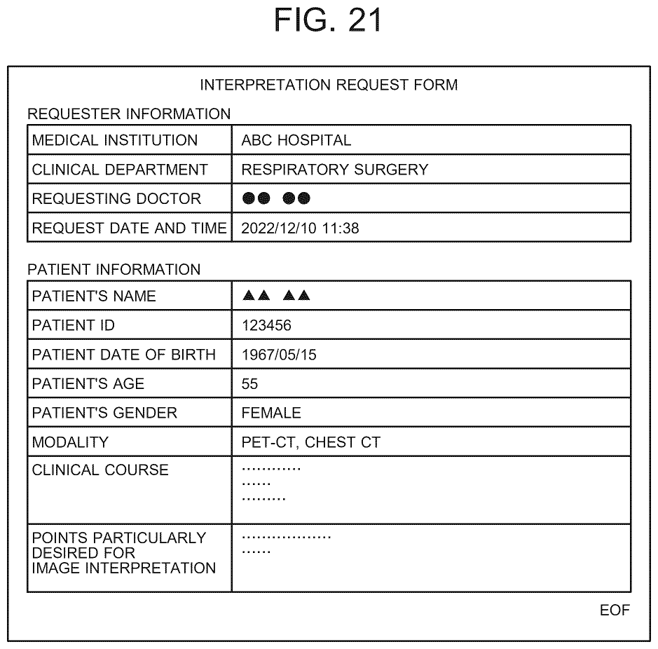

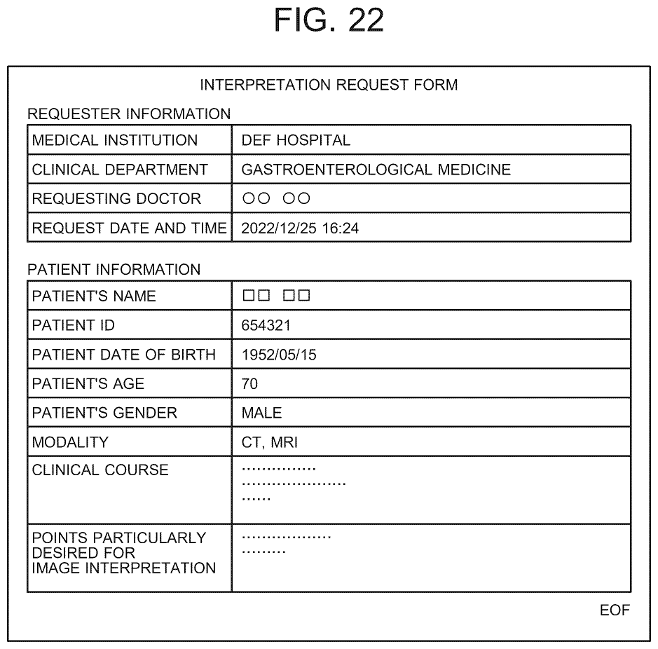

However, since the patient's name, the patient ID, the patient's date of birth, the clinical course, and the points particularly desired for image interpretation are information that may be the patient personal information, it may be arranged so that these may be additionally registered in an interpretation request form (see FIGS. 21 and 22), which will be described later, instead of in the image interpretation request registration screen.

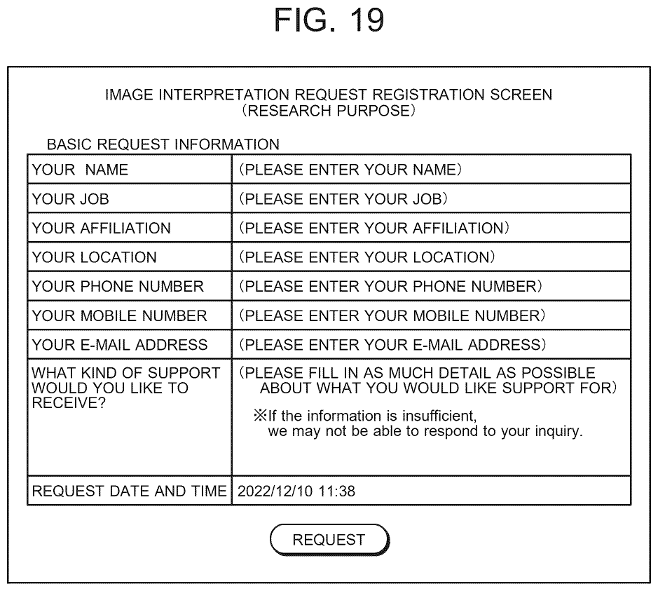

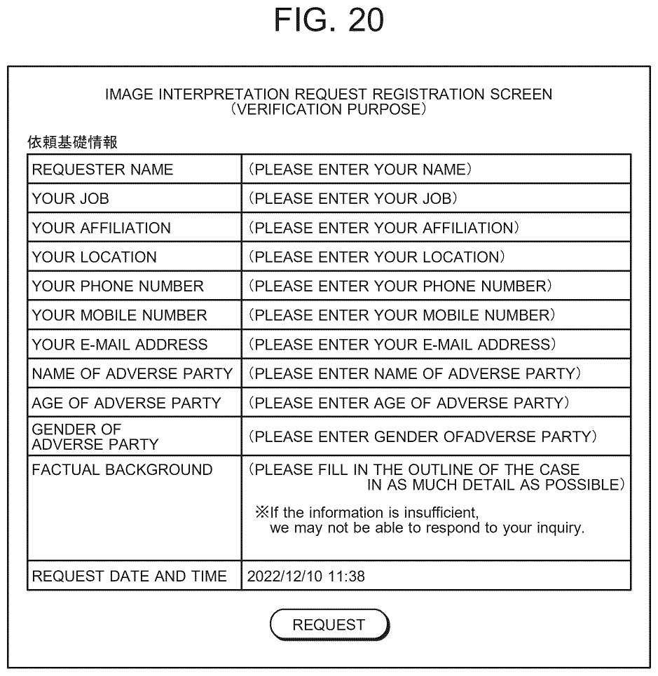

FIG. 19 shows an example of an image interpretation request registration screen for research purposes according to the present embodiment, and FIG. 20 shows an example of an image interpretation request registration screen for verification purposes according to the present embodiment. As shown in FIGS. 19 and 20, the contents of basic request information registered on the image interpretation request registration screen may differ depending on the purpose. In such case, the items managed in the request management table 116 illustrated in FIG. 3 may differ depending on the purpose.

In this way, the request basic information entered on the image interpretation request registration screen (including the request date and time) is input to the acceptance management server 110 as a character string, and is registered and managed in a predetermined record of the request management table 116 by the request management section 112 of the acceptance management server 110. At that time, the request management section 112 may generate a request ID and store it in the predetermined record. Furthermore, the requester ID may be entered by the requester by providing an entry field on the image interpretation request registration screen, or may be specified from the requester DB 142 managed by the user management server 140 based on the basic request information entered on the image interpretation request registration screen. Moreover, the acceptant ID may be left blank (Null) if the acceptant is undecided, and after the acceptant is determined, the acceptant ID may be specified from the acceptant DB 143 managed by the user management server 140.

Next, the payment processing request section 113 of the acceptance management server 110 accesses the user management server 140 based on the name of the medical institution 200, the clinical department, the name of the requesting doctor, and so forth, included in the request basic information, and inquires about information regarding the requester pre-registered in the requester DB 142 with respect to the corresponding medical institution 200 (steps S1109 to S1409). In response, the user management section 141 of the user management server 140 acquires information about the requester of the corresponding medical institution 200 by referring to the requester DB 142 (see FIG. 7) based on the name of the medical institution 200, the clinical department, the name of the requesting doctor, and so forth, notified from the acceptance management server 110, and notifies the acceptance management server 110 of the acquired information (steps S1410 to S1110).

Here, if the medical institution 200 that is the requester is not yet registered in the requester DB 142 (if this is the first request from the medical institution 200, for instance), additional steps where the acceptance management server 110 provides the requester client 210 with a screen for registering information about the medical institution 200, notifies the user management server 140 of the information entered on this screen, and newly registers the information notified by the user management section 141 of the user management server 140 in the requester DB 142 may be added.

Next, the payment processing request section 113 of the acceptance management server 110 requests the payment server 130 to execute a payment processing regarding the current request based on the payment information included in the information regarding the requester notified from the user management server 140 (steps S1111 to S1311). In this process, the payment information may be notified from the acceptance management server 110 to the payment server 130, or the information acquisition section 131 of the payment server 130 may access the user management server 140 and acquire the payment information.

Next, the payment processing section 132 of the payment server 130 requests the medical institution 200 to make a payment regarding the current request by providing a payment screen to the requester client 210 (steps S1312 to S2112). In this process, the acceptance management server 110 may automatically switch the access destination of the requester client 210 to the payment server 130 to guide the medical institution 200 to smoothly perform payment.

Next, the medical institution 200 executes the payment regarding the current request by inputting a predetermined operation according to the payment screen displayed on the web browser of the requester client 210 (steps S2113 to S1313). In this embodiment, the payment information such as debit account information and credit card information is managed in advance at the service providing site 100, and the requester (in this example, the medical institution 200) can use this managed payment information for payment. Therefore, it is possible to avoid the risk caused by requiring the requester to enter payment information every time a request is made, and it is possible to realize a safe transaction.

Next, upon confirming that the medical institution 200 has completed the payment procedure, the receipt issuing section 133 of the payment server 130 issues a receipt regarding the current request to the requester client 210 (step S1314). In response, the medical institution 200 can download (DL) the receipt from the payment server 130 as necessary using the requester client 210 (steps S1315 to S2115). Note that the receipt issued by the payment server 130 may be, for example, an electronic file such as a PDF (Portable Document Format). Furthermore, the service providing site 100 may mail a paper receipt to the medical institution 200 upon request. Thereafter, the payment processing section 132 of the payment server 130 notifies the acceptance management server 110 that the payment procedure by the medical institution 200 has been completed (steps S1316 to S1116).

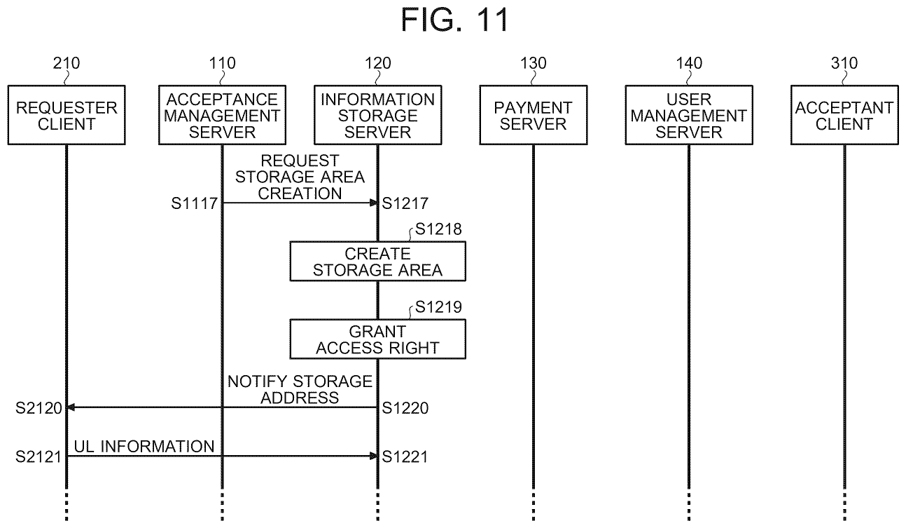

Next, as shown in FIG. 11, the storage area creation request section 114 of the acceptance management server 110 requests the information storage server 120 to create (including securing) a storage area 125 for securely storing information including highly confidential information such as the patient's name, the patient's date of birth, the medical images to be interpreted, and so forth (steps S1117 to S1217). In this process, the acceptance management server 110 may notify the information storage server 120 of part of the information regarding the requester (for example, the requester ID, the e-mail address, etc.).

Next, the storage area creation section 121 of the information storage server 120 creates (or secures) the storage area 125 accessible via the network 400 in the storage section 124 (step S1218). Subsequently, the access right granting section 122 of the information storage server 120 grants the medical institution 200 access rights to the created (or secured) storage area 125 (step S1219). For example, when the information storage server 120 is a cloud storage, the information storage server 120 may create a data box that is accessible via the network 400 (see step S1218), and grant the medical institution 200 (which may be the requester client 210) access rights to this data box. (see step S1219).

Next, the access right granting section 122 of the information storage server 120 notifies the medical institution 200 of an address (for example, a URL (Uniform Resource Locator)) for accessing the created (or secured) storage area 125 via the network 400 (steps S1220 to S2120). Note that the information storage server 120 may notify the medical institution 200 of the storage address using e-mail or the like.

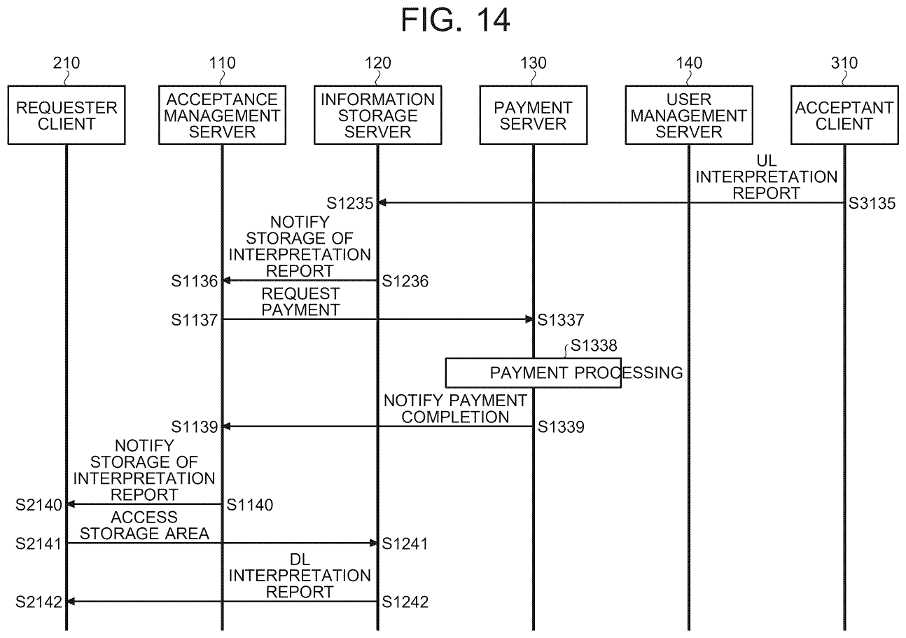

When the storage address is notified in this way, the medical institution 200 accesses the storage area 125 from the requester client 210 using the storage address, and uploads (UL) more detailed information regarding the request contents (hereinafter also referred to as request information) to the storage area 125 (steps S2121 to S1221). Note that the request information may also include information such as an electronic medical chart (for example, an electronic medical chart output as PDF). Furthermore, when the request information is stored in the storage area 125, it may be encrypted with a randomly generated password. The encryption of the request information may be executed in the requester client 210 or in the storage information management section 123 of the information storage server 120, for instance. The password used for the encryption may be appropriately notified to the medical institution 200 that uploaded the request information and/or the interpretation doctor 300 that accepted the request. For example, the password may also be notified when the storage address is notified in steps S1131 to S3131 in FIG. 13, which will be described later.

Here, the request information stored in the storage area 125 will be explained in detail with reference to FIGS. 21 and 22. FIGS. 21 and 22 are diagrams illustrating examples of the request information according to the present embodiment. Note that although FIGS. 21 and 22 show examples of the request information excluding medical images, the request information according to the present embodiment may include medical images to be interpreted.

As shown in FIGS. 21 and 22, the request information other than medical images may be stored in the storage area 125 as a file titled “Interpretation request form,” for example. The request information may include information such as “requester information,” “patient information,” “clinical course,” and “points particularly desired for image interpretation,” for instance.

The “requester information” may include information such as the name of the medical institution 200, the clinical department, the requesting doctor, and the date and time of the request, for instance. The “patient information” may include information such as the name, the date of birth, the age, the gender, etc., of the patient in addition to the information about the patient ID and the modality, for instance. Note that among the requester information and the patient information, the information included in the request basic information entered on the image interpretation request registration screen may be used from this information.

In addition, the “clinical course” may be information regarding healthcare interventions, and so forth, performed on the patient so far, and the “points particularly desired for image interpretation” may be information regarding points that the requesting doctor particularly wants the interpretation doctor to interpret.

The interpretation request form containing the above information may be stored in the storage area 125 as a file saved in a document format such as a text file, a word file or a PDF, or in an image format such as JPEG, GIF, TIFF or PNG, or in a video format such as MP4, for instance.

The request information may be uploaded to the storage area 125 using a registration information management screen provided by the storage information management section 123 of the information storage server 120, for instance. FIG. 23 is a diagram illustrating an example of the registration information management screen according to the present embodiment.

As shown in FIG. 23, the registration information management screen may include a column for selecting files to be uploaded to the reserved storage area 125, an “UPLOAD” button for uploading the selected files to the reserved storage area 125, a column displaying a list of files stored in the storage area 125, a “DOWNLOAD” button for individually downloading files selected from the list, and a “BATCH DOWNLOAD” button for downloading files stored in the storage area 125 all at once, for instance.

When the medical institution 200 uploads the request information to the storage area 125 allocated to itself, the medical institution 200 views the registration information management screen using the web browser function of the requester client 210, and selects files to be uploaded (an interpretation request form, one or more medical images, an audio file, etc.) on this registration information management screen, and then, click the “UPLOAD” button. Thereby, the selected files are uploaded to the information storage server 120 and stored in the designated storage area 125 by the storage information management section 123.

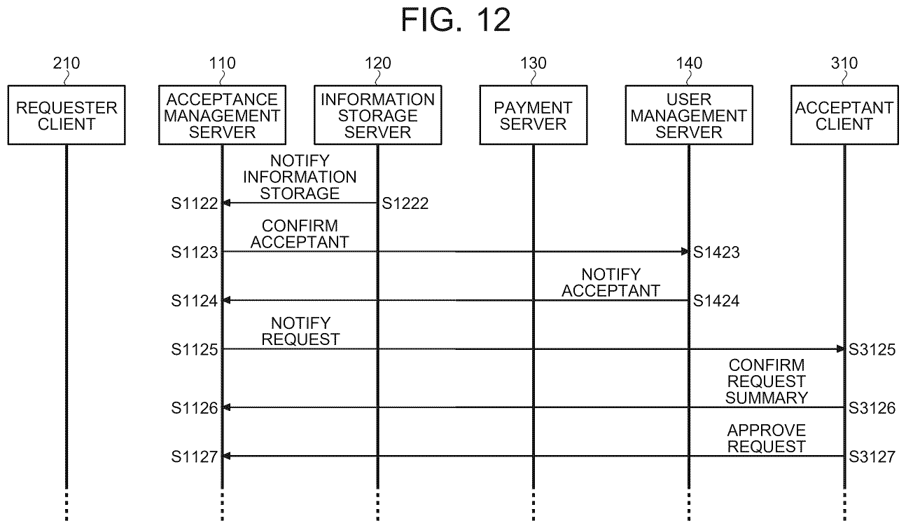

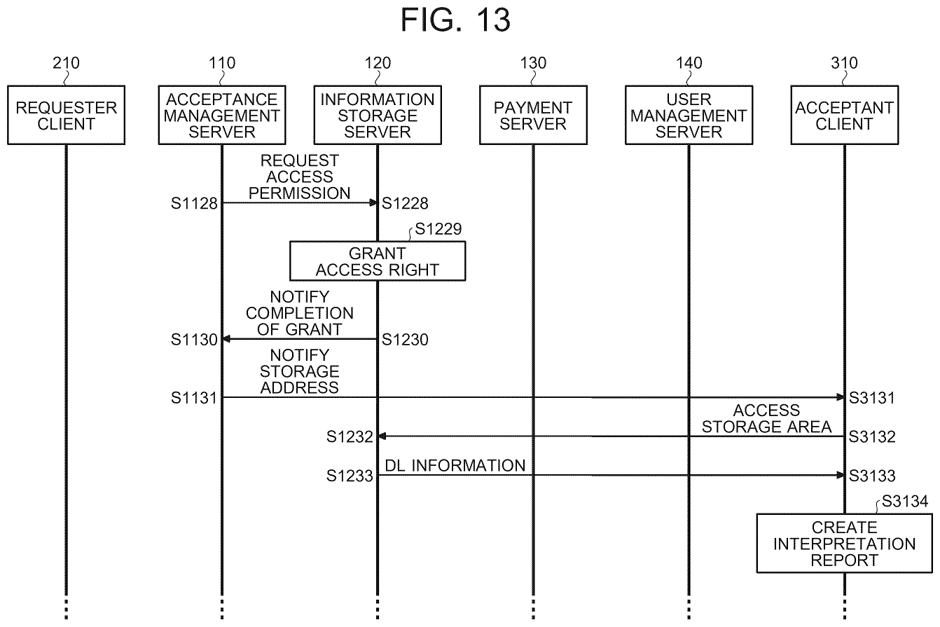

Return to explanation of the operation. As described above, as shown in FIG. 12, when the request information is stored in the storage area 125 in the storage section 124, the storage information management section 123 of the information storage server 120 notifies the acceptance management server 110 that the request information has been stored (steps S1222 to S1122).

Next, the information storage notification section 115 of the acceptance management server 110 inquires of the user management server 140 for information about the interpretation doctor who will be the acceptant (information about the acceptant) (steps S1123 to S1423). At that time, the information storage notification section 115 notifies the user management server 140 of the request basic information or a part thereof. In response, the user management section 141 of the user management server 140 identifies the interpretation doctor 300 who will be the acceptant of the current request (specifically, the information about the acceptant) by referring to the acceptant DB 143 based on the information notified from the acceptance management server 110 at the time of the inquiry, and notifies the acceptance management server 110 of at least the contact information (for example, e-mail address) of the information about the identified acceptant (steps S1424 to S1124).

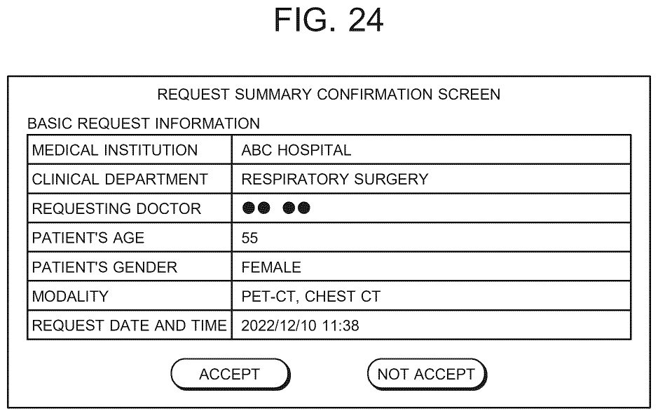

Next, the information storage notification section 115 of the acceptance management server 110 notifies the interpretation doctor 300 (specifically, his/her contact information) notified by the user management server 140 that the request has been made (step S1125 to S3125). For example, when the e-mail address is notified from the user management server 140 to the acceptance management server 110 as the contact information for the interpretation doctor 300, the information storage notification section 115 automatically sends the e-mail to the address to notify that the request has been made. The e-mail may include a URL for guiding the interpretation doctor 300, who is the acceptant, to the request summary confirmation screen (see FIG. 24) for checking the summary of the request contents.

Here, FIG. 24 is a diagram illustrating an example of the request summary confirmation screen according to the present embodiment. As shown in FIG. 24, the request summary confirmation screen may include an area where displays information (for example, the basic request information) for the acceptant (in this example, the interpretation doctor 300) to understand the overview of the request, buttons for the acceptant to select whether to accept or not accept the request. Such the request summary confirmation screen may be automatically generated by the information storage notification section 115 of the acceptance management server 110 when the medical institution 200 registers the basic request information using the requester client 210 (steps S2108 to S1108).

The interpretation doctor 300 who has been notified of the request checks the request summary confirmation screen via the notified URL using the acceptant client 310 (steps S3126 to S1126), and determines whether or not to he/she should accept the request. Then, in a case where the interpretation doctor 300 accepts the request, the interpretation doctor 300 approves the request by clicking the “ACCEPT” button on the request summary confirmation screen, for instance (steps S3127 to S1127). On the other hand, if the interpretation doctor 300 clicks the “NOT ACCEPT” button, the requester client 210 may be notified via the acceptance management server 110 that the request has not been accepted by the interpretation doctor 300.

As shown in FIG. 13, when the request is accepted by the interpretation doctor 300, the information storage notification section 115 of the acceptance management server 110 requests the information storage server 120 to grant the interpretation doctor 300 access rights to the secured storage area 125 (steps S1128 to S1228). At this time, the information storage notification section 115 notifies the information storage server 120 of at least the acceptant ID among the information about the acceptant. In response, the access right granting section 122 of the information storage server 120 grants the interpretation doctor 300 (for example, the acceptant ID assigned to the interpretation doctor 300) the access right to the storage area 125 (step S1229), and notifies the acceptance management server 110 that the granting of the access right has been completed (steps S1230 to S1130). At this time, the address (for example, the URL) of the storage area 125 may be notified again from the information storage server 120 to the acceptance management server 110.

Next, the information storage notification section 115 of the acceptance management server 110 notifies the acceptant client 310 of the interpretation doctor 300 who received the request of the address (for instance, the URL) for accessing the storage area 125 via the network 400 (steps S1131 to S3131). At this time, as described above, when the request information is encrypted, the password used for this encryption may also be notified.

Next, the interpretation doctor 300 accesses the registration information management screen (see FIG. 23) via the notified URL using the acceptant client 310 (steps S3132 to S1232), and downloads (DL) the file (the request information) stored in the storage area 125 (steps S3133 to S1233). Note that when the interpretation doctor 300 accesses the registration information management screen (see FIG. 23) using the acceptant client 310, a login authentication process using the acceptant ID, password, etc. may be performed similarly to the login authentication process (steps S1102 to S2107) performed for the requester (in this example, the medical institution 200), for instance.

In this way, the interpretation doctor 300 who has acquired the request information (the interpretation request form, the one or more medical images, the audio files, etc.) interprets one or more medical images according to the contents of the request and creates an interpretation report (step S3134). Note that the information processing device used to interpret the medical images is not limited to the acceptant client 310, and various information processing devices may be used.

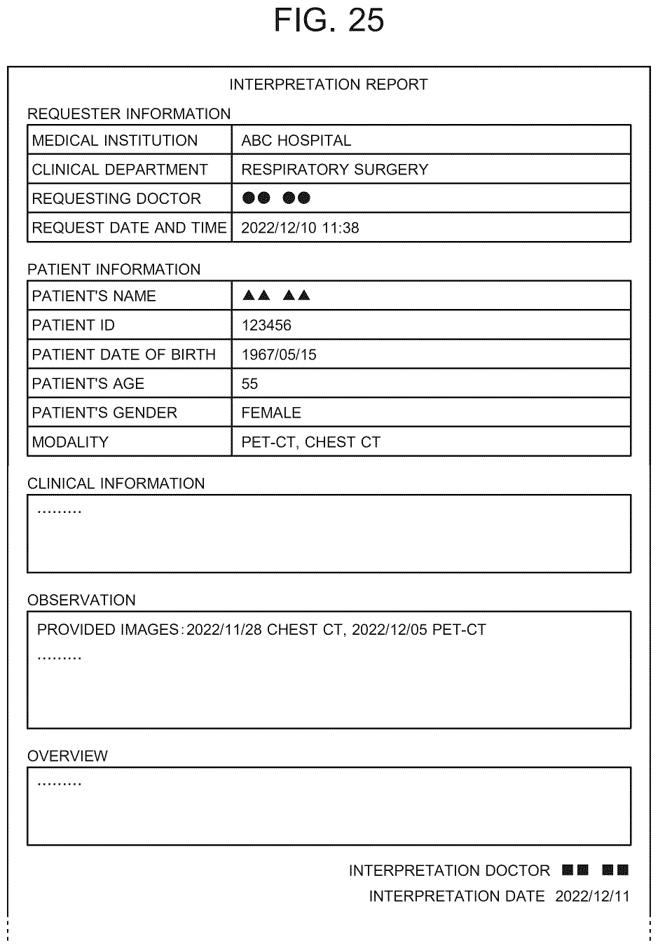

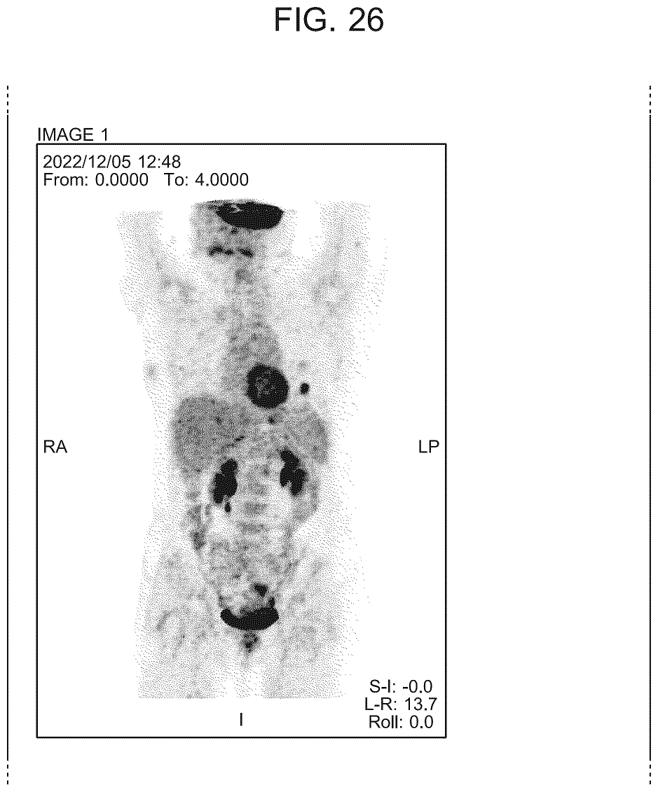

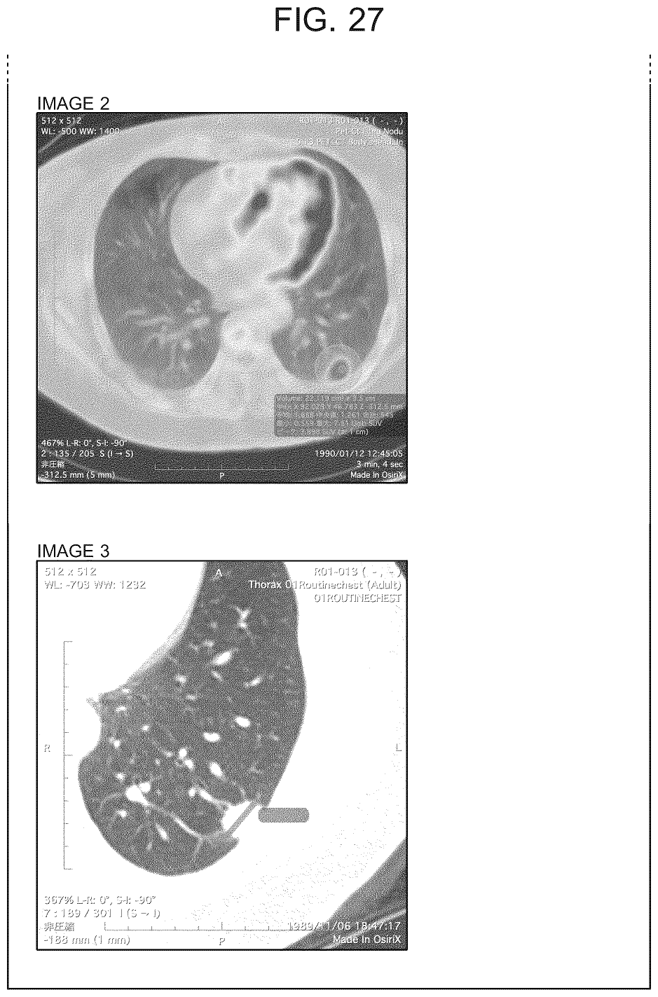

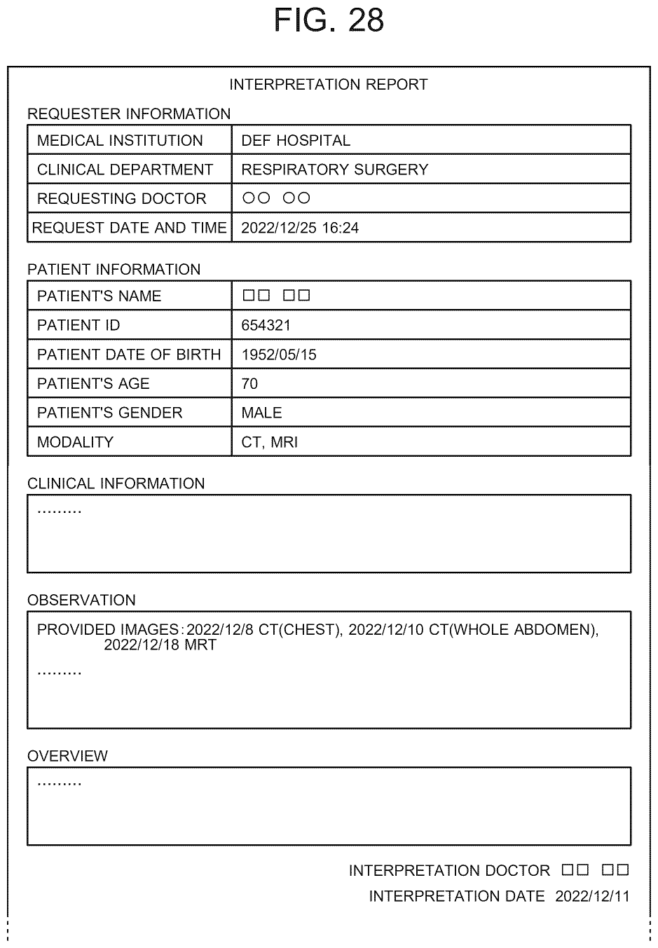

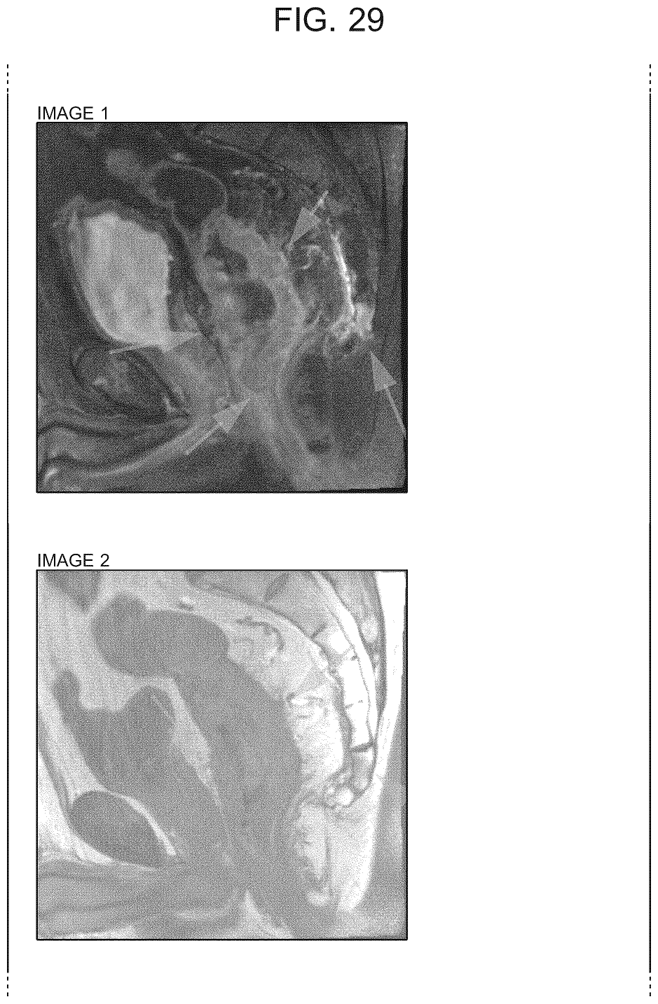

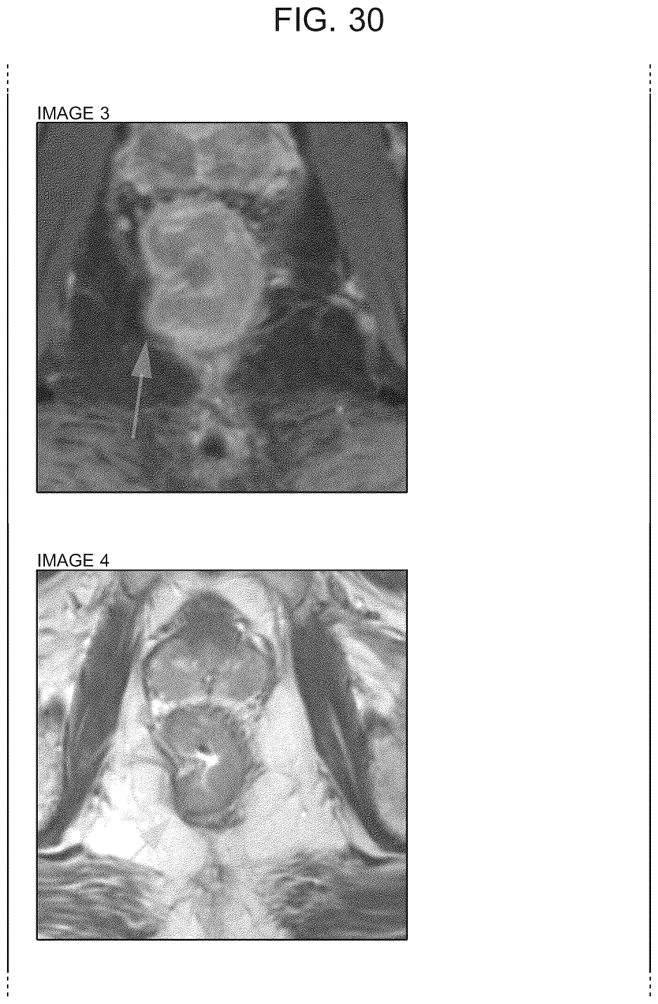

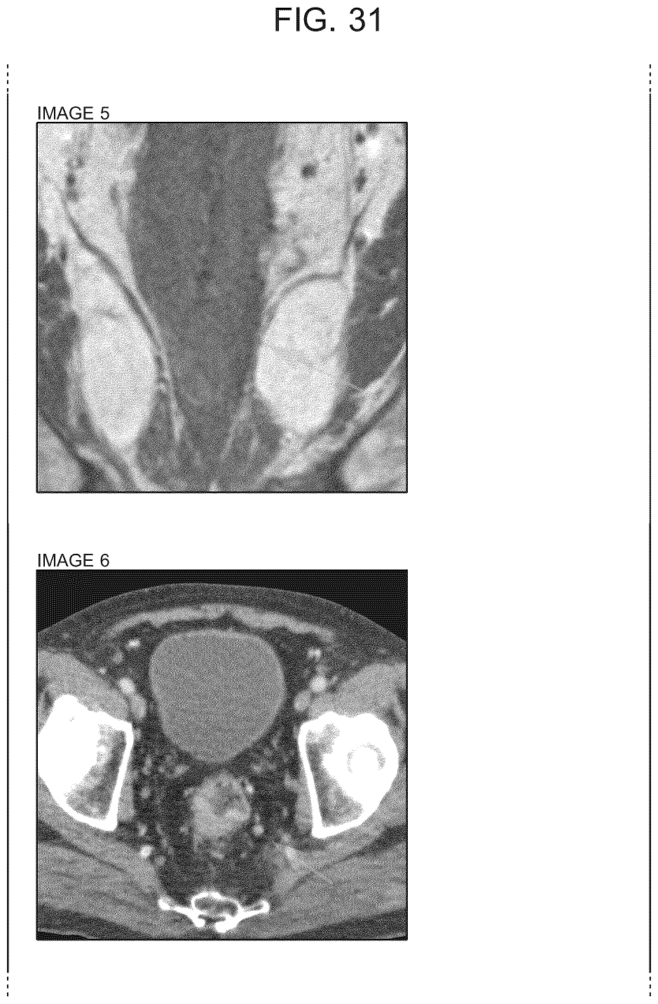

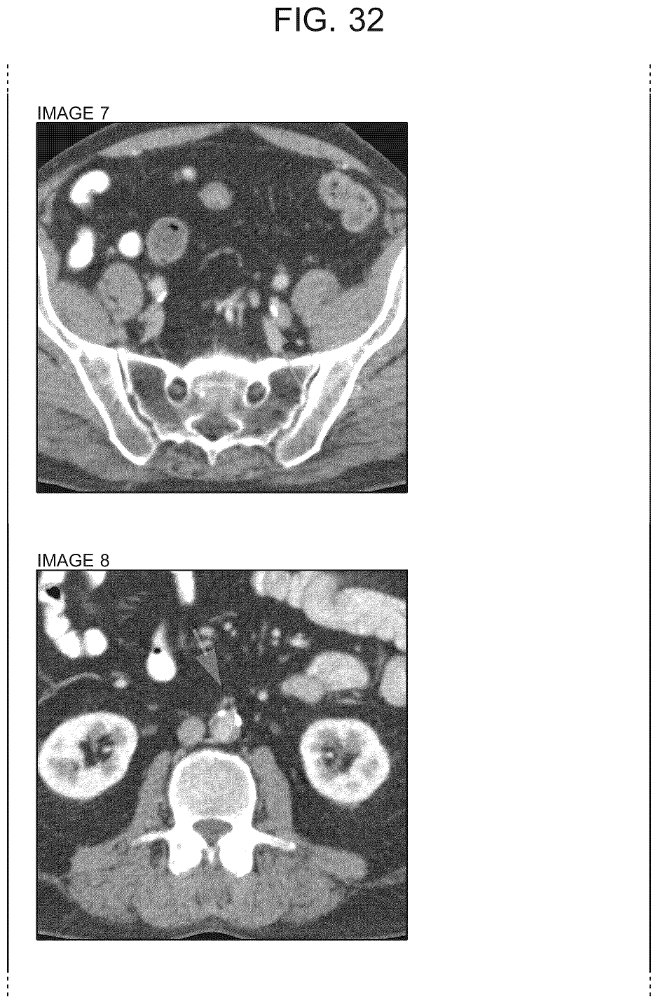

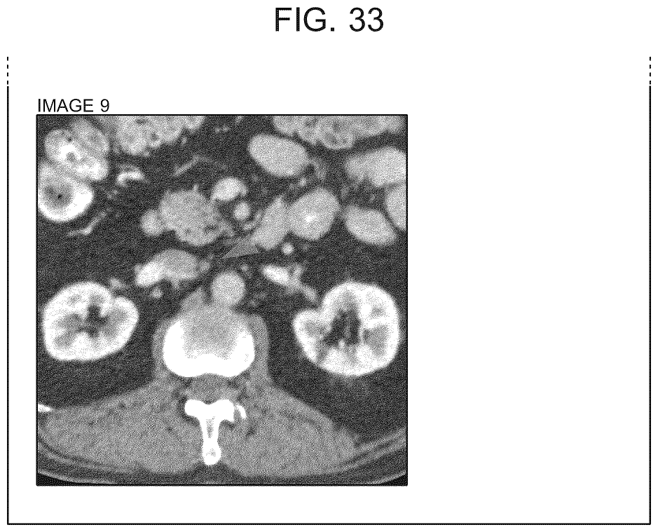

Here, the interpretation report created by the interpretation doctor 300 will be explained using a specific example. FIGS. 25 to 27 and FIGS. 28 to 33 are diagrams showing examples of image interpretation reports according to the present embodiment.

As shown in FIGS. 25 to 27 and FIGS. 28 to 33, the image interpretation report may include client information, patient information, a clinical course, an observation, and a summary. The client information, the patient information, and the clinical course may be cited from those included in the interpretation request form. The observation may be created by the interpretation doctor 300 as a result of interpreting the medical images based on the patient information, the clinical course, and so forth. The summary may be created as a result of an overall judgment including other information by the interpretation doctor 300.

Furthermore, if necessary, one or more medical images may be attached to the interpretation report to facilitate understanding of the interpretation report (particularly, the observation and the summary). These medical images may be edited with arrows, boxes, coloring/color-coding, etc. to indicate points of interest during image interpretation, points mentioned in the observation and the summary, and the like. Note that the interpreted medical images are not limited to being attached to the interpretation report, and may be stored in the storage area 125 as separate files, for instance.

Return to explanation of the operation. As mentioned above, when the interpretation report is created, as shown in FIG. 14, the interpretation doctor 300 accesses the registered information management screen (see FIG. 23) via the notified URL using the acceptant client 310, and uploads the created interpretation report to the storage area 125 secured in the storage section 124 of the information storage server 120 (steps S3135 to S1235). In response, the storage information management section 1123 of the information storage server 120 notifies the acceptance management server 110 that the interpretation report has been stored (steps S1236 to S1136). Note that when the interpretation report is stored in the storage area 125, the interpretation report may be encrypted using a randomly generated password. The encryption of the interpretation report may be executed in the acceptant client 310 or in the storage information management section 123 of the information storage server 120, for instance. The password used for the encryption may be appropriately notified to the interpretation doctor 300 who uploaded the interpretation report and/or the medical institution 200 that placed the request. For example, the password may also be notified at the time of the interpretation report storage notification in steps S1140 to S2140 in FIG. 14, which will be described later.

Next, the payment processing request section 113 of the acceptance management server 110 requests the payment server 130 to execute a payment processing to the interpretation doctor 300 (steps S1137 to S1337). In this process, the account information may be notified from the acceptance management server 110 to the payment server 130, or the payment server 130 may access the user management server 140 and obtain the account information. Upon receiving the payment request from the acceptance management server 110, the payment processing section 132 of the payment server 130 executes a payment (transfer) process for the amount set in the current request according to the payment information (step S1338). Then, after the payment processing is completed, the payment processing section 132 notifies the acceptance management server 110 that the payment processing has been completed (steps S1339 to S1139).

Furthermore, when the information storage notification section 115 of the acceptance management server 110 is notified from the information storage server 120 that the interpretation report has been stored in the storage area 125 (see steps S1236 to S1136), the information storage notification section 115 notifies the medical institution 200 that the interpretation report has been stored in the storage area 125 based on the contact information in the information regarding the requester (steps S1140 to S2140). At this time, as described above, if the interpretation report is encrypted, the password used for the encryption may also be notified.

Next, the medical institution 200 accesses the registration information management screen (see FIG. 23) using the requester client 210 (steps S2141 to S1241), and downloads the interpretation report stored in the storage area 125 (steps S1242 to S2142). Thereby, the medical institution 200 that is the requester obtains the interpretation report for the current request.

With the above operation, since it becomes possible to exchange the highly confidential information such as the patient personal information and the interpretation report between the requester (the medical institution 200) and the acceptant (the interpretation doctor 300) via the storage area 125 where information can be stored securely, it becomes possible to realize the secure remote image diagnosis comprehensive support system 1 in which information confidentiality is maintained. Then, according to the present embodiment, since the service providing site 100 can provide consistent support from receiving requests to payment and registration (delivery) of image interpretation reports, it becomes possible to significantly reduce the hurdles for doctors to participate in medical practice as image interpreters. As a result, it becomes possible to provide the remote image diagnosis comprehensive support apparatus, the method of comprehensive support for remote diagnostic imaging, and the program that enable more doctors to participate in medical care as image interpreters.

Furthermore, according to the present embodiment, by using the acceptance management service and the payment service provided by the service providing site 100, it is possible to significantly reduce the time and effort required for request/acceptance processes and accounting processes for the requester and the acceptant, whereby it becomes possible to significantly reduce intermediate costs such as personnel expenses and management expenses. Moreover, according to the present embodiment, the acceptant, such as the interpretation doctor 300, can directly contract with the requester, such as the medical institution 200, to receive the request, and therefore it is also possible to significantly reduce intermediate costs such as marketing costs.

Conventionally, a clinician or a medical institution wishing to request an image interpretation are required to register for a fixed-term contract with a professional image interpretation company which requires payment of initial fees and monthly usage fees, and there has been no such on-demand service where you can request an image interpretation only when you want. Furthermore, clinicians who request difficult-to-diagnose cases, complex cases, or the like, generally want to have an opportunity to select an interpretation doctor who specializes in such cases. However, due to problems such as there is an environment in which it is difficult for interpretation doctors to freely enter the field, and there is a situation in which a professional image interpretation company has the authorization to allocate works, it may not be possible for the clinicians to always request a desired image interpretation doctor, which makes diagnosis difficult.

In contrast, according to the present embodiment, the clinician, the medical institution 200 requesting the image interpretation, and the interpretation doctor 300 can work on an on-demand basis, so the interpretation requesting side and the image interpretation side can conduct marketing freely. Even in such case, it is possible to automatically perform accounting on both the interpretation requesting side and the image interpretation side, so it is possible to significantly reduce intermediate costs.

In the present embodiment, although the case has been exemplified in which the compensation is paid to the interpretation doctor 300 each time an interpretation report is stored in the storage area 125, that is, each time a request is received and fulfilled, payment of compensation to the interpretation doctor 300 is not limited to such arrangement. For example, the number of requests for each interpretation doctor 300 and each plan is managed per predetermined period (one month, for instance) in the acceptance management server 110, and an amount corresponding to the number of requests may be paid to each interpretation doctor 300 when or after a predetermined period has elapsed (for example, at the end of the month, or the like).

Moreover, in the present embodiment, although the case has been exemplified in which the medical institution 200 is billed for the amount according to the selected plan each time a request is made, the billing to the medical institution 200 is not limited to such arrangement. For example, the number of requests for each medical institution 200 and each plan is managed for each predetermined period (for example, one month) in the acceptance management server 110, and, an amount corresponding to the number of requests may be billed to each medical institution 200 when or after a predetermined period has elapsed (for example, at the end of the month, or the like).

### (Modification of First Embodiment)

In the above-described first embodiment, the case where the acceptant is an individual interpretation doctor 300 has been exemplified. Here, if the acceptant is a professional image interpretation company that has multiple interpretation doctors, for instance, work is required to allocate received requests to each interpretation doctor within the professional image interpretation company. In this modification, a configuration for automating an allocation of requests to each interpretation doctor within a professional image interpretation company will be described using an example.

An automatic allocation of requests to each interpretation doctor may be performed in the acceptant client 310, for instance. In such case, the acceptant client 310 may manage information (also referred to as interpretation doctor information) such as an identification number (also referred to as an interpretation doctor ID), a clinical department, a specialty (head, chest, abdomen, etc.), interpretation results (fields of medical images interpreted, number of interpretations, etc.), specialized modalities (X-ray, CT, PET-CT, MRI, etc.), applications used for image interpretation (Osirix, DICOM Library, IMAIOS DICOM Viewer, PostDICOM, Voxelx, FViewer, etc.) for each interpretation doctor, and based on the interpretation doctor information and the request information, an appropriate interpretation doctor may be automatically allocated to each request. For example, the acceptant client 310 may allocate requests in order of the interpretation doctor ID consisting of numbers, may allocate requests based on the specialty, may allocate requests based on the specialized modalities, or may allocate requests based on two or more items in the interpretation doctor information.

Downloading of the request information from the service providing site 100 may be executed from the acceptant client 310 of the professional image interpretation company, or may be executed from a client of an interpretation doctor to whom the request is allocated. When the downloading is executed from the client of the interpretation doctor, an access right to the storage area 125 may also be granted to this client.

Likewise, uploading of the interpretation report from the professional image interpretation company to the service providing site 100 may be executed from the acceptant client 310 of the professional image interpretation company, or may be executed from a client of an interpretation doctor to whom the request is allocated. When the uploading is executed from the client of the interpretation doctor, an access right to the storage area 125 may also be granted to this client.

Other configurations, operations and effects may be the same as those of the first embodiment described above, so detailed explanations will be omitted here.

### Second Embodiment

Next, a second embodiment of the present disclosure will be described in detail with reference to the drawings. In the followings, for clarity, descriptions will be given focusing on the differences from the remote image diagnosis comprehensive support system 1.

In the present embodiment, a case in which one or more requesters request interpretation of medical images for a plurality of acceptants is exemplified. In the following, as in the first embodiment, for clarity, a case where each of a plurality of acceptants is an individual interpretation doctor and the acceptants are medical institutions will be exemplified.

### (System Configuration)

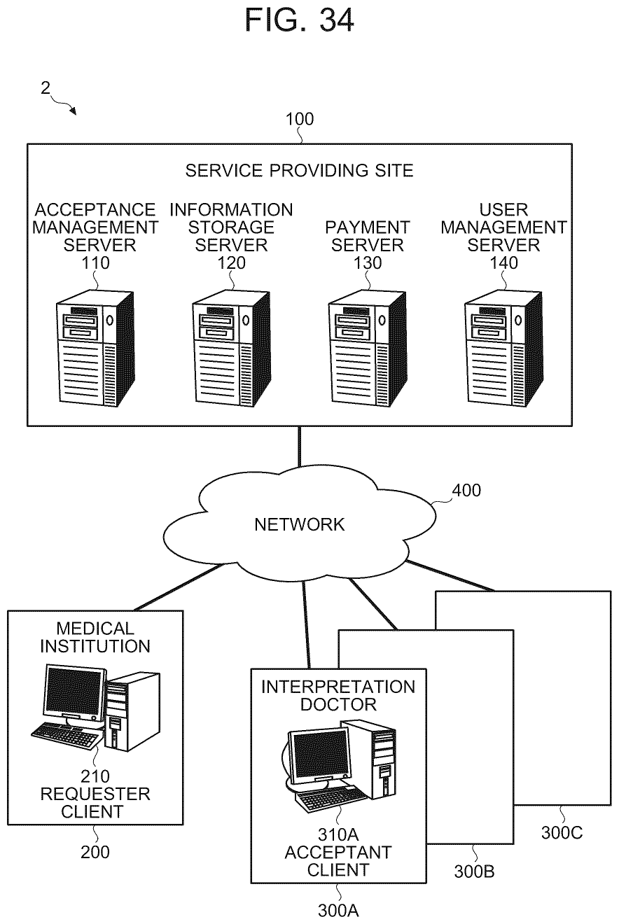

FIG. 34 is a schematic diagram illustrating a schematic configuration example of a remote image diagnosis comprehensive support system for realizing a remote image diagnosis comprehensive support service according to the present embodiment. As is clear from comparing FIG. 34 and FIG. 1, whereas the case explained about the remote image diagnosis comprehensive support system 1 according to the first embodiment shows that the relationship between the requester (for example, the medical institution 200) and the acceptant (for example, the interpretation doctor 300) was one-on-one, in a case of a remote image diagnosis comprehensive support system 2 according to the present embodiment, it will be explained that the relationship between the requester (for example, the medical institution 200) and acceptants (for example, interpretation doctors 300A to 300C) is one-to-many.

The configurations of the various servers 110, 120, 130 and 140 constituting the service providing site 100 according to the present embodiment and the requester client 210 of the medical institution 200 may be the same as those according to the first embodiment. Furthermore, the configurations of the acceptant clients 310A to 310C of the interpretation doctors 300A to 300C according to the present embodiment may be the same as the acceptant clients 310 of the interpretation doctor 300 according to the first embodiment.

### (Operation Example)

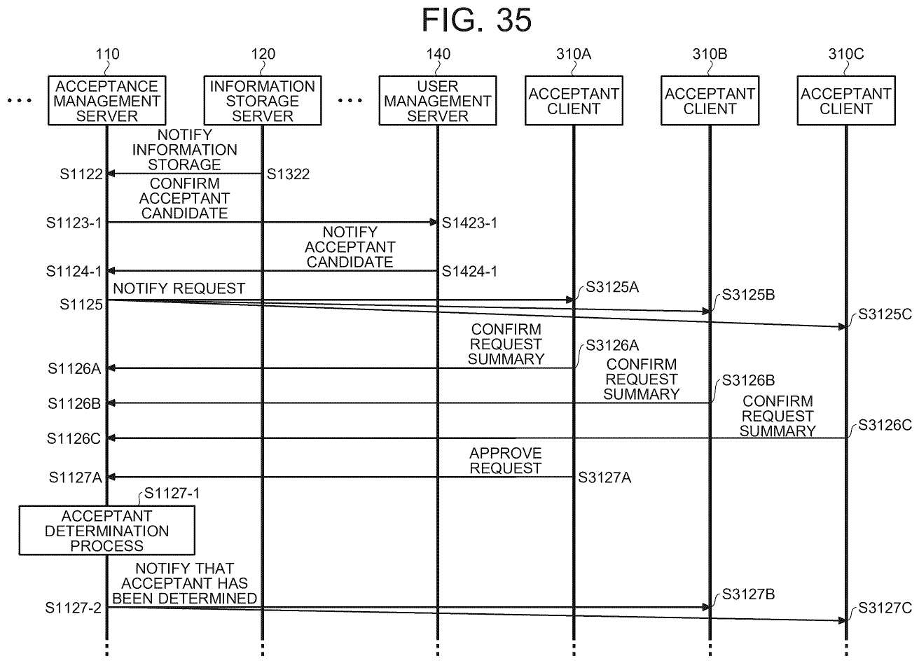

Next, an example of an operation of the remote image diagnosis comprehensive support system 2 according to the present embodiment will be described. FIG. 35 is a sequence diagram illustrating an example of an operation of the remote image diagnosis comprehensive support system according to the present embodiment. In the following description, steps similar to the operations of the remote image diagnosis comprehensive support system 1 according to the first embodiment will be cited and redundant descriptions will be omitted. Note that, in the operation example of the remote image diagnosis comprehensive support system 2 according to the present embodiment, the single acceptant client 310 in the first embodiment is replaced with a plurality of requester clients 310A to 310C.

In the present embodiment, first, by performing operations similar to those described using FIGS. 9 to 11 in the first embodiment, the request information is stored in the storage area 125 secured within the storage section 124 of the information storage server 120. Then, as shown in FIG. 35, the storage information management section 123 of the information storage server 120 notifies the acceptance management server 110 that the request information has been stored (step S1222 to S1122).

Next, the information storage notification section 115 of the acceptance management server 110 inquires of the user management server 140 for information on one or more interpretation doctors who are candidates for the acceptants (information about the acceptants) (steps S1123-1 to S1423-1). In this process, similarly to steps S1123 to S1423 in FIG. 12, the information storage notification section 115 notifies the user management server 140 of the request basic information or a part thereof. In response, the user management section 141 of the user management server 140 identifies one or more interpretation doctors 300A to 300C who are acceptant candidates for the current request (specifically, the information about the acceptants) by referring to the acceptant DB 143 based on the information notified from the acceptance management server 110 at the time of the inquiry, and notifies the acceptance management server 110 of at least the contact information (for example, e-mail address) of each piece of information regarding the one or more identified acceptant candidates (steps S1424 to S1124).

Next, the information storage notification section 115 of the acceptance management server 110 notifies each of the one or more interpretation doctors 300A to 300C notified from the user management server 140 (specifically, their contact information) that the request has been made (steps S1125 to S3125A, S3125B and S3125C). Note that the request summary confirmation screen to which each of the one or more interpretation doctors 300A to 300C is guided via the notification may be similar to the one illustrated in FIG. 24.

Each of the one or more interpretation doctors 300A to 300C who have been notified of the request confirms the request summary confirmation screen via the notified URL using the acceptant clients 310A to 310C (steps S3126A to S1126A, S3126B to S1126B, and S3126C to S1126C), and determines whether or not to he/she should accept the request. When accepting the request, the interpretation doctor (in this example, the interpretation doctor 300A) approves the request by clicking the “ACCEPT” button on the request summary confirmation screen, for instance (steps S3127A to S1127A). Note that FIG. 35 illustrates a case where the interpretation doctor 300A first approves the request.

Next, the information storage notification section 115 of the acceptance management server 110 identifies the interpretation doctor (in this case, the interpretation doctor 300A) who first approved the request from among the one or more acceptant candidates, and determines this interpretation doctor 300A as the acceptant (step S1127-1). Then, the information storage notification section 115 notifies the interpretation doctors 300B and 300C other than the interpretation doctor 300A determined as the acceptant that the acceptant has been determined (steps S1127-2 to S3127B/S3127C).

In the present embodiment, after one acceptant is determined in this way from among one or more acceptant candidates, by performing operations similar to those described using FIGS. 13 and 14 in the first embodiment, the interpretation report created by the interpretation doctor 300A is provided to the medical institution 200 that is the requester via the storage area 125 secured in the storage section 124 of the information storage server 120.

As described above, according to the present embodiment, even when there are multiple acceptant candidates, since highly confidential information such as patient personal information, an interpretation report, and so forth, can be exchanged between the requester (the medical institution 200) and the acceptant (the interpretation doctor 300A, 300B or 300C) via the storage area 125 where information can be stored securely, it is possible to realize the secure remote image diagnosis comprehensive support system 2 in which information confidentiality is maintained.

Other configurations, operations and effects may be the same as those of the first embodiment, so detailed explanations will be omitted here.

### (Modification of Second Embodiment)

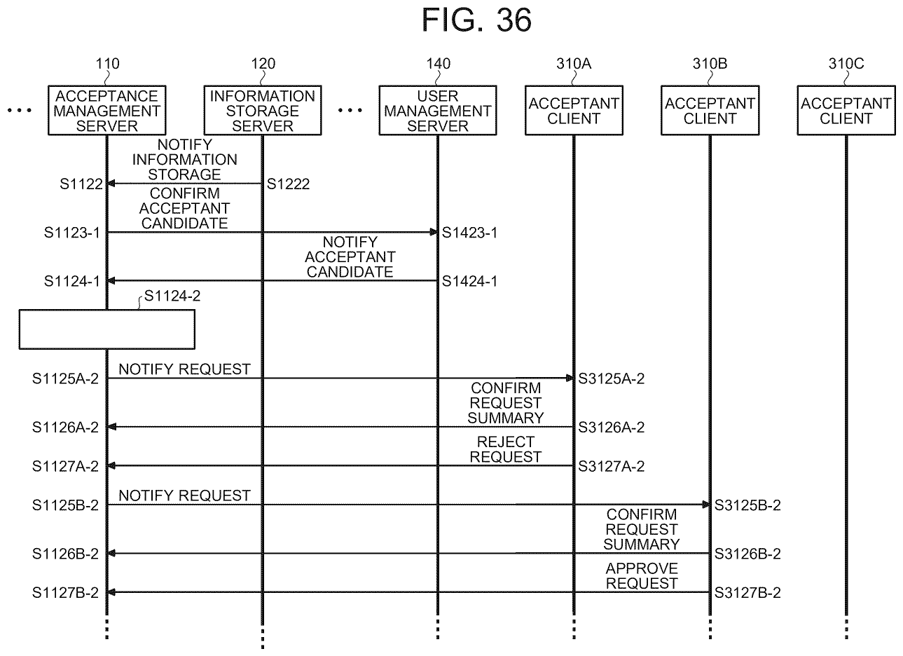

Next, a modification of the second embodiment, that is, another form in which one or more acceptant candidates exist for one requester, will be described with reference to the drawings. The remote image diagnosis comprehensive support system according to this modification may be similar to the remote image diagnosis comprehensive support system 2 illustrated in FIG. 34. However, in this modification, the operation illustrated in FIG. 35 may be replaced with an operation illustrated in FIG. 36.

As shown in FIG. 36, in the present embodiment, first, by performing operations similar to those described using FIGS. 9 to 11 in the first embodiment, the request information is stored in the storage area 125 secured within the storage section 124 of the information storage server 120. Then, by performing operations similar to those described using steps S1222 to S1124-1 in FIG. 35 in the second embodiment, the acceptance management server 110 is notified of at least the contact information (e.g., e-mail address) of each of the information regarding the one or more interpretation doctors 300A to 300C (specifically, information about the acceptants) who are acceptant candidates for the current request.

Next, the information storage notification section 115 of the acceptance management server 110 determines the priority order for one or more of the interpretation doctors 300A to 300C notified from the user management server 140 (step S1124-2). Note that, the priority order may be determined by the information storage notification section 115 based on the request content, acceptable field, acceptance history, compatible medical image analysis software, and so on, for each acceptant managed in the acceptant DB 143, or it may be determined in advance and managed in the acceptant DB 143, for instance.

After the priority order is determined, the information storage notification section 115 first notifies the interpretation doctor with the highest priority (in this example, the interpretation doctor 300A) that the request has been made (steps S1125A-2 to S3125A-2). The request summary confirmation screen to which the interpretation doctor 300A is guided via the notification may be similar to the one illustrated in FIG. 24.

The interpretation doctor 300A who has been notified of the request checks the request summary confirmation screen via the notified URL using the acceptant client 310 (steps S3126A-2 to S1126A-2), and determines whether or not to he/she should accept the request. Then, in a case where the interpretation doctor 300 does not accept the request, the interpretation doctor 300 notifies the acceptance management server 110 that the request will not be accepted by clicking the “NOT ACCEPT” button on the request summary confirmation screen (steps S3127A-2 to S1127A-2), for instance.

If the acceptant candidate with one higher priority rejects the request, the information storage notification section 115 of the acceptance management server 110 notifies the interpretation doctor with the next highest priority (in this example, the interpretation doctor 300B) of the request (steps S1125B-2 to S3125B-2). In response, the interpretation doctor 300B checks the request summary confirmation screen via the notified URL using the acceptant client 310B (steps S3126B-2 to S1126B-2) and determines whether or not to he/she should accept the request, and when accepting the request, approves the request by clicking the “ACCEPT” button on the request summary confirmation screen (steps S3127B-2 to S1127B-2), for instance.

When one acceptant is determined from the one or mor acceptant candidates, in the present embodiment, by performing operations similar to those described using FIGS. 13 and 14 in the first embodiment, the interpretation report created by the interpretation doctor 300B is provided to the medical institution 200 that is the requester via the storage area 125 secured in the storage section 124 of the information storage server 120.

As described above, according to the present embodiment, since it becomes possible to exchange highly confidential information such as the patient personal information and the interpretation report between the requester (the medical institution 200) and the acceptant (the interpretation doctor 300A, 300B or 300C) via the storage area 125 where information can be stored securely even if there are multiple acceptant candidates, it becomes possible to realize the secure remote image diagnosis comprehensive support system 1 in which information confidentiality can be maintained.

Other configurations, operations and effects may be the same as those of the first or second embodiment, so detailed explanations will be omitted here.

### Third Embodiment

Next, a third embodiment of the present disclosure will be described in detail with reference to drawings. In the following description, for clarity, the same configurations, operations, and effects as those of the first or second embodiment or the modification thereof will be cited and redundant descriptions will be omitted.

In the above-described embodiments and the modification thereof, the case where particularly highly confidential information such as patient personal information, or the like, is exchanged between the requester and the acceptant in the medical field was exemplified. In response, in the present embodiment, a case where the above-described embodiments are generalized and applied to the e-commerce market will be exemplified.

Here, generalization may mean that a requester is a general consumer (regardless of professional or amateur), an acceptant is a specialist, and an request content is general. By generalization, it becomes possible to apply a request support system according to the present embodiment to various categories of e-commerce in which various data such as image data, video data, audio data, and document data may be exchanged between a requester and an acceptant, such as requests for lessons or coaching on musical instruments such as piano, guitar, violin, etc., requests for lessons or coaching for sports such as golf, baseball, soccer, billiards, classical ballet, and social dance, etc., requests for lessons or coaching in painting, calligraphy, karaoke, etc., requests for foreign language lessons such as English conversation and English speech, etc., requests for lyrics, composition, arrangement, etc., requests for production, editing, and proofreading of novels and comics, etc., requests for production and debug of app, and so forth, for instance.

Conventionally, when requesting proofreading or editing of novels, comics, or songs (hereinafter also referred to as productions), the author saves the productions on a storage medium such as a CD-ROM (Compact Disc-Read Only Memory) or USB (Universal Serial Bus) memory and delivers the storage medium to the editor directly or via mail, or sends the productions as e-mail attachments. However, in such a method, there was a problem that unreleased productions could be lost or unintentionally made public due to loss or theft of storage media, leakage via e-mail, etc.

Furthermore, in recent years, so-called online lessons, in which the above-mentioned lessons and coaching in sports, music, etc. are provided using video calling, are becoming popular. However, online lessons have the problem that teachers and students are tied to the same amount of time. Therefore, there is a growing demand for so-called offline lessons, in which students send demonstration videos of sports, music, etc., paintings or calligraphy works they have made, or copies or photographs thereof to their teachers, and can receive lessons and coaching from their teachers who watch them later. However, in offline lessons, as same as the requests for proofreading and editing of productions, it has been a problem that there is a possibility that productions that the person does not wish to make public could be made public unintentionally due to loss or theft of storage media, leakage via e-mail, etc.

Therefore, the present embodiment is created in view of the above problems, and the purpose of the present disclosure is to provide a request support apparatus, a request support method, and a program that can enhance the confidentiality of information when requesting offline lessons or coaching.

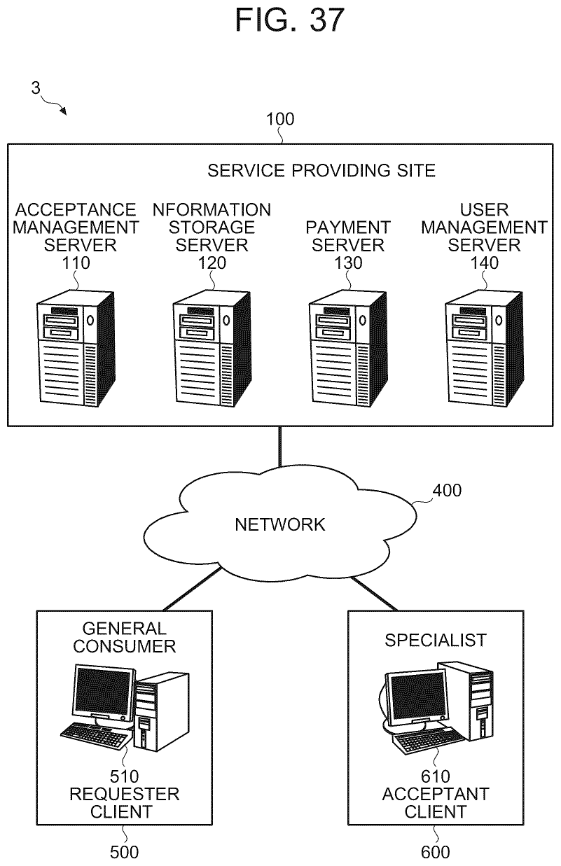

FIG. 37 is a diagram illustrating a schematic configuration example of a request support system for realizing a request support service according to the present embodiment. As shown in FIG. 37, the request support system 3 according to the present embodiment, in the same configuration as the remote image diagnosis comprehensive support system 1 illustrated in FIG. 1, the medical institution 200 is replaced with a general consumer 500, and the interpretation doctor 300 is replaced with a specialist 600, for instance. The general consumer 500 can access the service providing site 100 using the requester client 510, and the specialist 600 can access the service providing site 100 using the acceptant client 610. The requester client 510 and the acceptant client 610 may be similar to the requester client 210 and the acceptant client 310 according to the first embodiment.

In the above configuration, the general consumer 500 who is a requester registers a request to the specialist 600 on the service providing site 100 using a requester client 510, similar to the medical institution 200 in the first or second embodiment. On the other hand, the specialist 600 who is the acceptant accepts the request from the specialist 600 and stores a deliverable in the service providing site 100 using the acceptant client 610. Then, the general consumer 500 accesses the service providing site 100 using the requester client 510 and obtains the registered deliverable.

The flow from registration of a request from the general consumer 500 to the service providing site 100 to acquisition of the deliverable from the service providing site 100 by the general consumer 500 via reception of the request by the specialist 600 and storage of the deliverable on the service providing site 100 may be the same as in the first or second embodiment or the modifications thereof.

However, in the present embodiment, medical images targeted for image interpretation in the first embodiment may be replaced with data that the specialist 600 analyzes and provides advice (also called request data) such as video data of musical instruments or sports (for example, golf swings), audio data of singing voices or performances, document data such as novels, comics, programs, or the like.

Furthermore, each item stored in the request management table 116, the requester DB 142, and the acceptant DB 143 illustrated in the first embodiment may be changed as appropriate depending on categories of request contents.

As described above, even when generalized, since information that the requester wants to keep confidential, such as highly confidential information like personal information and information like video data, can be exchanged between the requester (the general consumer 500) and the acceptant (the specialist 600), it is possible to realize the secure request support system 3 in which the confidentiality of information can be maintained.

Other configurations, operations and effects may be the same as those of the first or second embodiment or the modifications thereof, so detailed explanations will be omitted here.

### <Hardware Configuration>

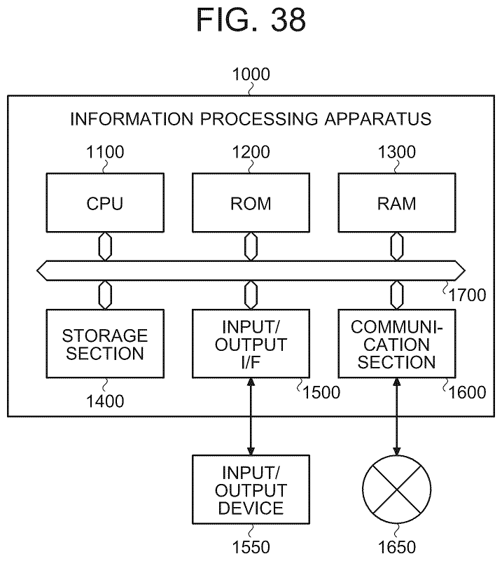

The acceptance management server 110, the information storage server 120, the payment server 130, the user management server 140, the requester clients 210 and 510, the acceptant clients 310, 310A, 310B, 310C and 610, and so forth, according to the above-described embodiments and the modifications thereof can be realized by an information processing apparatus 1000 having a configuration as shown in FIG. 38, for example. FIG. 38 is a hardware configuration diagram showing an example of the information processing apparatus 1000 that realizes the functions of the acceptance management server 110, the information storage server 120, the payment server 130, the user management server 140, the requester clients 210 and 510, the acceptant clients 310, 310A, 310B, 310C and 610, and so forth. The information processing apparatus 1000 includes a CPU 1100, a ROM (Read Only Memory) 1200, a RAM (Random Access Memory) 1300, a storage section 1400, an input/output interface (I/F) 1500, and a communication section 1600. Each section of the information processing apparatus 1000 is connected via a bus 1700.

The CPU 1100 operates based on a program stored in the ROM 1200 or the storage section 1400, and controls each section. For example, the CPU 1100 loads programs stored in the ROM 1200 or the storage section 1400 into the RAM 1300, and executes processes corresponding to various programs.

The ROM 1200 stores boot programs such as a BIOS (Basic Input Output System) that are executed by the CPU 1100 when the information processing apparatus 1000 is started, programs that depend on the hardware of the information processing apparatus 1000, and the like.

The storage section 1400 is a computer-readable storage medium that non-transitorily records programs executed by the CPU 1100, data used by the programs, and the like. Specifically, the storage section 1400 is a storage medium that stores a program for executing each operation according to the present disclosure, which is an example of program data.

The communication section 1600 is an interface for connecting the information processing apparatus 1000 to an external network 1650 (the Internet, for instance). For example, the CPU 1100 receives data from other devices or transmits data generated by the CPU 1100 to other devices via the communication section 1600.

The input/output I/F 1500 is an interface for connecting the input/output device 1550 and the information processing apparatus 1000. For example, the CPU 1100 receives data from an input device such as a keyboard or a mouse via the input/output I/F 1500. Furthermore, the CPU 1100 transmits data to an output device such as a display, a speaker, or a printer via the input/output I/F 1500. Moreover, the input/output I/F 1500 may function as a media interface that reads programs and the like recorded on a predetermined storage medium.

For example, when the information processing apparatus 1000 functions as the acceptance management server 110, the information storage server 120, the payment server 130, the user management server 140, the requester clients 210 and 510, the acceptant clients 310, 310A, 310B, 310C and 610, and so forth, according to the above-described embodiments, the CPU 1100 of the information processing apparatus 1000 realizes the functions of the acceptance management server 110, the information storage server 120, the payment server 130, the user management server 140, the requester clients 210 and 510, the acceptant clients 310, 310A, 310B, 310C and 610, and so forth, by executing a program loaded onto the RAM 1300. Furthermore, the storage section 1400 stores programs and the like according to the present disclosure. Note that although the CPU 1100 reads program data from the storage section 1400 and executes it, as another example, the CPU 1100 may obtain these programs from other devices via the external network 1650.

### REFERENCE SIGNS LIST

1,2 Remote image diagnosis comprehensive support system
3 Request support system
100 Service providing site
110 Acceptance management server
111 Request acceptance section
112 Request management section
113 Payment processing request section
114 Storage area creation request section
115 Information storage notification section
116 Request management table
120 Information storage server
121 Storage area creation section
122 Access right granting section
123 Storage information management section 123
124 Storage section
125 Storage area
130 Payment server
131 Information acquisition section
132 Payment processing section
133 Receipt issuing section
140 User management server
141 User management section
142 Requester database
143 Acceptant database
144 User authentication section
200 Medical institution
210, 510 Requester client
300, 300A, 300B, 300C Interpretation doctor
310, 310A, 310B, 310C, 610 Acceptant client
400 Network
500 General consumer
600 Specialist

1-9. (canceled)

10. A method of comprehensive support for remote diagnostic imaging that requests interpretation of medical images in DICOM (digital imaging and communication in medicine) format to an external party via the public internet, comprising:
accepting a request for an image interpretation from a requester, and guiding the requester to register basic request information including a name of the requester, a clinical department that diagnoses a medical image, a name of a requesting doctor, a name of a patient, an age of a patient, a gender of a patient, and a modality;
managing the request by registering the basic request information registered by the requester in a request management table;
executing payment by the requester regarding the request;
creating a storage area for securely storing highly confidential request information including the medical image in a storage section of a server on the public internet which is different from a server storing the request management table;
granting an access right to the storage area to the requester;
notifying the requester of an address for accessing the storage area;
storing the request information uploaded by the requester in the storage area;
granting an access right to the storage area to an acceptant who accepted the request;
notifying the acceptant of the address for accessing the storage area;
downloading the request information in the storage area to the acceptant on demand from the acceptant;
storing an interpretation report regarding the medical image uploaded by the acceptant in the storage area;
executing transfer to the acceptant uploaded the interpretation report; and
downloading the interpretation report in the storage area to the requester on demand from the requester, wherein
the payment includes managing the number of one or more requests placed by the requester within a predetermined period, and billing the requester for the amount to be paid for the one or more requests placed by the requester within the predetermined period in total, and
the transfer includes managing the number of one or more request accepted by the acceptant within a predetermined period, and transferring the amount to be paid for the one or more requests accepted by the acceptant within the predetermined period to the acceptant in total.

11. The method of comprehensive support for remote diagnostic imaging according to claim 10, wherein
the storing of the request information uploaded by the requester in the storage area includes encrypting the request information uploaded by the requester using a randomly generated first password, storing the encrypted request information in the storage area, and notifying the acceptant of the first password, and
the storing of the interpretation report regarding the medical image uploaded by the acceptant in the storage area includes encrypting the interpretation report uploaded by the acceptant using a randomly generated second password, storing the encrypted interpretation report in the storage area, and notifying the requester of the second password.

12. The method of comprehensive support for remote diagnostic imaging according to claim 10, further comprising issuing a receipt to the requester when the requester executes the payment regarding the request.

13. The method of comprehensive support for remote diagnostic imaging according to claim 10, further comprising:
managing in advance first information including payment information for executing the payment with respect to the requester, and managing in advance second information including account information for executing the transfer with respect to the acceptant, wherein
the payment is executed using the managed payment information, and
the transfer is executed using the managed account information.

14. The method of comprehensive support for remote diagnostic imaging according to claim 10, wherein
the acceptant includes a plurality of interpretation doctors, and the method further comprising:
managing interpretation doctor information for each of the interpretation doctors including at least one of a clinical department, a specialty, interpretation results, a specialty modality, and an application used for interpretation interpretations, and
determining an assignment of the accepted request among the plurality of the interpretation doctors based on the request information downloaded from the storage area and the managed interpretation doctor information.

15. A method of comprehensive support for remote diagnostic imaging that requests interpretation of medical images in DICOM (digital imaging and communication in medicine) format to an external party via the public internet, comprising:
accepting a request for an image interpretation from a requester, and guiding the requester to register basic request information including a name of the requester, a clinical department that diagnoses a medical image, a name of a requesting doctor, a name of a patient, an age of a patient, a gender of a patient, and a modality;
managing the request by registering the basic request information registered by the requester in a request management table;
executing payment by the requester regarding the request;
creating a storage area for securely storing highly confidential request information including the medical image in a storage section of a server on the public internet which is different from a server storing the request management table;
granting an access right to the storage area to the requester;
notifying the requester of an address for accessing the storage area;
storing the request information uploaded by the requester in the storage area;
determining a priority order of a plurality of acceptance candidates;
notifying the plurality of acceptant candidates that the request has been made in order of the priority order;
determining an acceptance candidate who approves the request first as an acceptant who accepts the request;
granting an access right to the storage area to the acceptant;
notifying the acceptant of the address for accessing the storage area;
downloading the request information in the storage area to the acceptant on demand from the acceptant;
storing an interpretation report regarding the medical image uploaded by the acceptant in the storage area;
executing transfer to the acceptant uploaded the interpretation report; and
downloading the interpretation report in the storage area to the requester on demand from the requester.

16. The method of comprehensive support for remote diagnostic imaging according to claim 15, wherein
the storing of the request information uploaded by the requester in the storage area includes encrypting the request information uploaded by the requester using a randomly generated first password, storing the encrypted request information in the storage area, and notifying the acceptant of the first password, and
the storing of the interpretation report regarding the medical image uploaded by the acceptant in the storage area includes encrypting the interpretation report uploaded by the acceptant using a randomly generated second password, storing the encrypted interpretation report in the storage area, and notifying the requester of the second password.

17. The method of comprehensive support for remote diagnostic imaging according to claim 15, further comprising issuing a receipt to the requester when the requester executes the payment regarding the request.

18. The method of comprehensive support for remote diagnostic imaging according to claim 15, further comprising:
managing in advance first information including payment information for executing the payment with respect to the requester, and managing in advance second information including account information for executing the transfer with respect to the acceptant, wherein
the payment is executed using the managed payment information, and
the transfer is executed using the managed account information.

19. The method of comprehensive support for remote diagnostic imaging according to claim 15, wherein
the acceptant includes a plurality of interpretation doctors, and the method further comprising:
managing interpretation doctor information for each of the interpretation doctors including at least one of a clinical department, a specialty, interpretation results, a specialty modality, and an application used for interpretation interpretations, and
determining an assignment of the accepted request among the plurality of the interpretation doctors based on the request information downloaded from the storage area and the managed interpretation doctor information.

20. A non-transitory computer readable medium that retains a program for operating a processor which requests interpretation of medical images in DICOM (digital imaging and communication in medicine) format to an external party via the public internet, the program including instructions which, when executed by the processor, cause the processer to perform operations comprising:
accepting a request for an image interpretation from a requester, and guiding the requester to register basic request information including a name of the requester, a clinical department that diagnoses a medical image, a name of a requesting doctor, a name of a patient, an age of a patient, a gender of a patient, and a modality;
managing the request by registering the basic request information registered by the requester in a request management table;
executing payment by the requester regarding the request;
creating a area storage for securely storing highly confidential request information including the medical image in a storage section of a server on the public internet which is different from a server storing the request management table;
granting an access right to the storage area to the requester;
notifying the requester of an address for accessing the storage area;
storing the request information uploaded by the requester in the storage area;
granting an access right to the storage area to an acceptant who accepted the request;
notifying the acceptant of the address for accessing the storage area;
downloading the request information in the storage area to the acceptant on demand from the acceptant;
storing an interpretation report regarding the medical image uploaded by the acceptant in the storage area;
executing transfer to the acceptant uploaded the interpretation report; and
downloading the interpretation report in the storage area to the requester on demand from the requester, wherein
the process of executing the payment causes the processor to perform managing the number of one or more requests placed by the requester within a predetermined period, and billing the requester for the amount to be paid for the one or more requests placed by the requester within the predetermined period in total, and
the process of executing the transfer causes the processor to perform managing the number of one or more request accepted by the acceptant within a predetermined period, and transferring the amount to be paid for the one or more requests accepted by the acceptant within the predetermined period to the acceptant in total.

21. A non-transitory computer readable medium that retains a program for operating a processor which requests interpretation of medical images in DICOM (digital imaging and communication in medicine) format to an external party via the public internet, the program including instructions which, when executed by the processor, cause the processer to perform operations comprising:
accepting a request for an image interpretation from a requester, and guiding the requester to register basic request information including a name of the requester, a clinical department that diagnoses a medical image, a name of a requesting doctor, a name of a patient, an age of a patient, a gender of a patient, and a modality;
managing the request by registering the basic request information registered by the requester in a request management table;
executing payment by the requester regarding the request;
creating a storage area for securely storing highly confidential request information including the medical image in a storage section of a server on the public internet which is different from a server storing the request management table;
granting an access right to the storage area to the requester;
notifying the requester of an address for accessing the storage area;
storing the request information uploaded by the requester in the storage area;
determining a priority order of a plurality of acceptance candidates;
notifying the plurality of acceptant candidates that the request has been made in order of the priority order;
determining an acceptance candidate who approves the request first as an acceptant who accepts the request;
granting an access right to the storage area to the acceptant;
notifying the acceptant of the address for accessing the storage area;
downloading the request information in the storage area to the acceptant on demand from the acceptant;
storing an interpretation report regarding the medical image uploaded by the acceptant in the storage area;
executing transfer to the acceptant uploaded the interpretation report; and
downloading the interpretation report in the storage area to the requester on demand from the requester.
